(19) United States
(12) Patent Application Publication
(10) Pub. No.: US 2012/0108791 A1
(43) Pub. Date: May 3, 2012

(54) Rotamer Libraries and Methods of Use Thereof

(76) Inventors: Roland Dunbrack, Philadelphia, PA (US); Maxim Shapovalov, Glenside, PA (US)

(21) Appl. No.: 13/263,886

(22) PCT Filed: Apr. 20, 2010
(86) PCT No.: PCT/US10/31751

§ 371 Date: Nov. 10, 2011

Related U.S. Application Data
(60) Provisional application No. 61/170,908, filed on Apr. 20, 2009.

(51) Int. Cl. C40B 30/02; C40B 40/10; C07K 14/00; C40B 50/02

(52) U.S. Cl. 530/350; 506/24; 506/18; 506/8

(57) ABSTRACT

Rotamer libraries and methods of use thereof are provided.

This application claims priority under 35 U.S.C. §119(e) to U.S. Provisional Patent Application No. 61/170,908, filed on Apr. 20, 2009. The foregoing application is incorporated by reference herein.

This invention was made with government support under Grant Nos. P20 GM76222 and R01 GM84453 awarded by the National Institutes of Health. The government has certain rights in the invention.

### FIELD OF THE INVENTION

This invention relates generally to the fields of protein structure prediction and protein modeling and design.

### BACKGROUND OF THE INVENTION

Several publications and patent documents are cited in the foregoing specification in order to more fully describe the state of the art to which this invention pertains. The disclosure of each of these citations is incorporated by reference herein.

Prediction of the structures of proteins and protein complexes has a long history. Because of the complexity of the problem, most efforts have been restricted to certain aspects of the process, such as remote homologue identification and sequence-structure alignment (for a review, see Dunbrack, R. L. (2006) Curr. Opin. Struct. Biol., 16:374-384), loop structure prediction (Fiser et al. (2000) Prot. Science, 9:1753-1773; Tosatto et al. (2002) Protein Eng., 15:279-286; Jacobson et al. (2004) Proteins, 55:351-367; Zhang et al. (2004) Protein Sci., 13:391-399; Fernandez-Fuentes et al. (2005) Proteins, 60:746-57), and side-chain conformation prediction (Mendes et al. (2001a) J. Comp. Aided. Mol. Design, 15:721-740; Xiang and Honig (2001) J. Mol. Biol., 311:421-430; Desmet et al. (2002) Proteins, 48:31-43; Liang and Grishin (2002) Protein Sci., 11:322-331; Canutescu and Dunbrack (2003) Protein Sci., 12:963-972; Peterson et al. (2004) Protein Sci., 13:735-751; Xu, J. (2005) Rapid protein side-chain packing via tree decomposition, 9th Annual International Conference on Research in Computational Molecular Biology (RECOMB). 423-439). While ab initio prediction of protein structures has finally become a realistic option for small proteins (Bradley et al. (2005) Science, 309:1868-1871; Wu et al. (2007) BMC Biol., 5:17), it is still the case that the best structure prediction can be obtained when a homologous structure of the target protein is available. Similarly, programs are available for ab initio docking of small ligands to proteins (Lorber and Shoichet (2005) Curr. Top. Med. Chem., 5:739-749; Meiler and Baker (2006) Proteins, 65:538-548) and ab initio docking of two proteins (Fernandez-Recio et al. (2005) Proteins, 60:308-313; Schueler-Furman et al. (2005) Proteins, 60:187-194.).

Proteins may also be designed by determining a sequence that is likely to fold into a given structure in a particular environment (Jones (1994) Protein Eng., 3:567-574; Dahiyat et al. (1997) J. Mol. Biol., 4:789-796; Kortemme et al. (2004) Nat. Struct. Mol. Biol., 11:371-379). In protein design, packing, hydrophobic interactions, and hydrogen bonding patterns may be optimized by sampling of side chain conformations given a fixed backbone in a fixed environment (another protein or a ligand or DNA, for instance).

A particular aspect of this invention is a rotamer library, which may be defined as a set of protein side-chain conformations given by their mean dihedral angles and their variances and their associated frequencies of observation in experimentally determined structures. Rotamer libraries are used in all aspects of protein structure prediction and protein design to sample and score the most likely conformations of side chains.

### SUMMARY OF THE INVENTION

In accordance with the instant invention, backbone-dependent rotamer libraries and methods of making the same are provided. In a particular embodiment, the methods of making a backbone-dependent rotamer library comprise (a) determining rotamer probabilities as a function of the mainchain dihedrals φ and ψ by using von Mises kernels in adaptive kernel density estimates; (b) determining χ angle means and variances by using non-parametric kernel regression estimates; and (c) determining the backbone-dependent probability distributions of non-rotameric degrees of freedom using a combination of data-adaptive kernel density estimates for the side-chain degree of freedom and query-dependent kernel density estimates for the backbone degrees of freedom. In another embodiment, the methods comprise a Bayesian prior comprising the product of a χ₁-rotamer-backbone-dependent density estimate and backbone-independent conditional probabilities for the other degrees of freedom. In still another embodiment, the methods are performed with the assistance of a computer and the results outputted to the user in any desired manner (e.g., a print out or a display) and/or stored in memory. In yet another embodiment, at least part of the dataset used to generate the rotamer library is determined experimentally.

In accordance with another aspect of the instant invention, methods for generating an optimized structure of at least one polypeptide are provided. In a particular embodiment, the methods comprise the steps of (a) providing the backbone structure of the at least one polypeptide; (b) utilizing the rotamer library of the instant invention to establish a group of potential rotamers for at least one variable residue position in the polypeptide; and (c) analyzing the interaction of each of the rotamers obtained in step b) with at least part or all of the remainder of the polypeptide structure, thereby generating an optimized structure of the side chains of at least one amino acid of at least one polypeptide. In a particular embodiment, the method comprises the coordinates of a ligand (e.g., a proteinaceous or non-proteinaceous ligand) in step (a) and the resultant optimized structure of the polypeptide is in the presence of the ligand. In yet another embodiment, at least one variable residue position has rotamers from at least two different amino acid side chains. The methods may be employed to determine the optimal amino acid sequence of the polypeptide for binding (particularly increased binding) a ligand or second polypeptide. The methods may be performed with the assistance of a computer and the results outputted to the user in any desired manner (e.g., a print out or a display) and/or stored in memory. In yet another embodiment, the backbone structure may be determined experimentally. In still another embodiment, the optimized polypeptide may be tested experimentally for modulated binding of the ligand or second polypeptide compared to wild-type or the polypeptide prior to mutation.

In accordance with yet another aspect of the instant invention, methods for determining whether an alteration in a first polypeptide modulates the binding of the first polypeptide to a second polypeptide are provided. In a particular embodiment, the methods comprise (a) providing a set of structure coordinates for the amino acid residues of the first and second polypeptides; (b) modeling the interaction between the first and second polypeptides; (c) utilizing the rotamer library of the instant invention to establish a group of potential rotamers for at least one variable residue position in the first polypeptide, thereby producing a set of structure coordinates that define an altered first polypeptide; (d) modeling the interactions of the altered first polypeptide with the second polypeptide; and (e) determining whether the alteration modulates the interaction between the altered first polypeptide and said second polypeptide compared to the first polypeptide and the second polypeptide. In certain embodiment, the modulation of the interaction is a modulation (e.g., increase) in the avidity or binding affinity of the polypeptides. In a particular embodiment, one of the polypeptides is an antibody and the other protein is an antigen or epitope, particularly one recognized by the antibody. The methods may be performed with the assistance of a computer and the results outputted to the user in any desired manner (e.g., a print out or a display) and/or stored in memory). In yet another embodiment, the structure coordinates may be determined experimentally. In still another embodiment, the altered first polypeptide is tested experimentally for modulated binding of the second polypeptide compared to the first polypeptide and/or wild-type.

### BRIEF DESCRIPTION OF THE DRAWINGS

FIG. 1 provides graphs of kernel density estimates of p(φ,ψ|r) for the three χ₁ rotamers of serine, g⁺, trans, and g⁻ (left to right). Dots represent input data points.

FIG. 2 provides graphs of kernel density estimates of p(r|φ,ψ) for the three χ₁ rotamers of serine, g⁺, trans, and g⁻ (left to right). Dots represent input data points.

FIG. 3 is an image of the homology model of the C-terminal regulatory domain of human cystathionine β synthase, built with MolIDE and SCWRL, with the instant invention rotamer library. Side chains contacting the ligand S-adenosyl methionine are shown in stick representation.

FIG. 4 is an image of MolIDE2 with CBS-BD as a target. Structures checked in the hit list (upper left) are structurally aligned as shown in the resulting sequence alignment (lower left) and the interactive structure viewer (middle right). Blocks of structurally aligned residues are in blue. The sequence of the query has been added to the structure alignment (lower left) using a hidden Markov model (HMM) built from the structure alignment. A list of ligands in the checked entries appears upper right, and the PFAMs they interact with are given. Those entries checked in the ligand window contain the desired adenosine analogs; their residue interactions are highlighted in the sequence alignment. These structures are also highlighted in the ligand tree.

FIG. 5 provides a flowchart of the steps in SCWRL4 side-chain conformation prediction.

FIG. 6 provides examples of k-Dimensional Oriented Polytopes (kDOPs). Left panel: examples of kDOPs in the plane (k=2,3,4) and in three dimensions (k=3,4). Right panel: overlap test for kDOP A and kDOP B. The objects enclosed within the kDOPs may clash if the condition shown is satisfied.

FIG. 7 is a graph of SCWRL4 van der Waals potential. In this figure, x=d/Rᵢⱼ and θ=0.8254.

FIG. 8 is a schematic of hydrogen bond potential. Variables are defined in the text.

FIG. 9 provides a tree decomposition as generalization of biconnected component decomposition. At left, the graph used in the SCWRL3 paper is shown along with its biconnected component decomposition. At right, a tree decomposition of the same graph is shown. Residues c and d and residue h illustrate conditions 2 and 3 of a tree decomposition being satisfied, shown below the tree decomposition.

FIG. 10 demonstrates the effect of SCWRL4 features on differences in SCWRL3 and SCWRL4 accuracy. The accuracy shown is average absolute accuracy, which covers all side-chain dihedral angles. “Atomic radii”=use of optimized radii; “Interpolation”=interpolation of rotamer library probabilities and dihedral angles; “Local BB”=adding interaction between side chain and atoms N,HN of residue i−1 and C,O of residue i+1, previously neglected; “P=98%”=reading in top 98% of probability from rotamers sorted in descending order of frequency (previously 90%); “Hbonds”=new hydrogen bond potential; “New RL”=new rotamer library; “FRM”=Flexible rotamer model; “Tuning of parameters”=tuning of FRM parameters and rotamer library weight.

FIG. 11 is a graph demonstrating the improvement in accuracy due to inclusion of crystal neighbors. The accuracy figures shown reflect the differences in average absolute accuracy described in the text. No change was observed for Cys.

FIG. 12 provides graphs depicting the accuracy vs. percentile of electron density. Conditional accuracy for each dihedral degree of freedom is shown as a function of electron density percentile, using a sliding window of 20%.

FIG. 13 provides Table 1 depicting the accuracy of SCWRL4.

FIG. 14 provides Table 2 depicting the improvement of SCWRL4 over SCWRL3.

### DETAILED DESCRIPTION OF THE INVENTION

Rotamer libraries (see, e.g., Dunbrack, R. L. Curr. Opin. Struct. Biol. (2002) 12:431-440) are used extensively in many protein structure prediction and design applications from many research groups, and form the basis of side-chain prediction programs such as SCWRL (Canutescu and Dunbrack (2003) Protein Sci., 12:963-972). Rotamers are low-energy conformations of organic moieties, such as side chains of proteins, that are separated by high-energy barriers from other such conformations. Most degrees of freedom in protein side chains are “rotameric” in this way in the sense of being discrete conformations separated by high barriers. A few types of side chains have at least one “non-rotameric” dihedral degree of freedom, usually exhibiting only one broad, asymmetric peak in a probability density distribution. Certain rotameric states are higher in energy than others because of steric interactions with neighboring atoms. Electrostatic interactions including hydrogen bonds also affect side-chain energies. These interactions can be “backbone-independent” (i.e., not depending on the conformation of the local backbone of the residue) or “backbone-dependent” (i.e., depending on the local backbone conformation as determined by the backbone dihedral angles φ and ψ). Backbone-dependent rotamer libraries provide mean dihedral angles and their variances and the frequencies of protein side-chain conformations (i.e., rotamers) as a function of the backbone dihedrals φ and ψ. Both frequencies and mean angles and variances depend on the local backbone conformation (Dunbrack and Cohen (1997) Protein Sci., 6:1661-1681; Dunbrack and Karplus (1993) J. Mol. Biol., 230:543-574; Dunbrack and Karplus (1994) Nature Struct. Biol., 1:334-340; Dunbrack, R. L. (2006) Curr. Opin. Struct. Biol., 16:374-384).

Previous versions of the backbone-dependent rotamer library were based on a simple histogram analysis with k-nearest neighbors (Dunbrack and Karplus (1993) J. Mol. Biol., 230:543-574) or a histogram analysis with a Bayesian prior (Dunbrack and Cohen (1997) Protein Sci., 6:1661-1681; Dunbrack, R. L. (2006) Curr. Opin. Struct. Biol., 16:374-384). The latter has been extensively used in protein structure prediction and protein design by many research groups. The mean dihedral angles and sometimes their variances are used to sample conformations of side chains in proteins during structure prediction and design. The frequencies are used as part of an overall scoring function by taking the negative natural logarithm of each frequency multiplied by some constant as an energy term along with van der Waals, hydrogen bonding, and other energy terms. In some programs, such as Rosetta (Rohl et al. (2004) Methods Enz., 383:66-93), energy minimization of the entire structure is performed, requiring first derivatives of all the energy terms with respect to the backbone degrees of freedom φ and ψ.

The previous rotamer libraries have several significant drawbacks. First, the frequencies derived from the Bayesian histogram analysis are very bumpy as a function of φ and ψ. This is especially problematic for structure refinement methods that require smooth and differentiable energy surfaces (such as Rosetta). But it is also a problem in that some frequencies were badly estimated because of small amounts of data. This feature is characteristic of histogram methods. Second, the mean angles and their variances are even bumpier, with large changes in mean angles due to small amounts of data near some φ, ψ grid points. This means that many of them were badly determined and would produce sharp movements during a full energy minimization of the structure. Finally, the non-rotameric degrees of freedom were treated as if they were rotameric with only 3-6 discrete, non-overlapping bins to cover 180° or 360° (depending on the side-chain type). These are not adequate to perform continuous energy minimizations of these degrees of freedom. Due to the widespread use of the backbone-dependent rotamer library in protein structure prediction and protein design, an improved rotamer library that rectifies these problems will have a broad impact.

The rotamer library of the instant invention is derived from a more rigorously filtered data set and using entirely different and novel statistical methods. Indeed, the rotamer libraries of the instant invention comprise at least one, at least two, at least three, at least four, at least five, or all of the following characteristics.

First, the new rotamer library takes advantage of the much larger dataset that is available now than at the time of the past libraries and uses electron density calculations to remove highly dynamic side chains (or protein segments) that have uncertain conformations or coordinates. Software to measure electron densities at atomic coordinate positions for side-chains in any set of structures for which experimental structure factors are available (Shapovalov and Dunbrack (2007) Proteins, 66:279-303). For example, electron density maps available from the Uppsala Electron Density Server (EDS) (Kleywegt, et al. (2004) Acta Crystallogr. D Biol. Crystallogr., 60:2240-2249) may be used. Further, the PISCES server (Wang and Dunbrack (2003) Bioinformatics, 19:1589-1591) handles the problem of choosing a set of structures with resolution and R-factors better than fixed cutoffs and with mutual sequence identity less than a set value. One advantage of PISCES is that it can take an input list of PDB entries or chains and choose a subset of these using input single and pairwise criteria. Therefore, the list of X-ray crystallographic entries that have structure factor data available (about half of X-ray entries) can be used, and PISCES can be used to determine an appropriate subset of these. For instance, at 1.7 Å and 50% sequence identity cutoff, it is possible to generate a list of 2,123 chains with available structure factors. If the availability of structure factors is not considered, the list would have a size of 2,196. For comparison, the most recent rotamer library (2002) used 850 chains with resolution better than 1.7 Å and maximum 50% mutual sequence identity.

Second, the frequencies of rotamers as a function of the backbone dihedrals φ and ψ have been calculated using adaptive kernel density estimates (Abramson (1982) Annals Stat., 10:1217-1223) using a von Mises kernel function (Hall et al. (1987) Biometrika, 74:751-762). The rotamer library probabilities can be derived using Bayes' rule from the Ramachandran densities of each rotamer type, r:

$$p ( r \vert φ , ψ ) = \frac{p ( φ , ψ \vert r ) p ( r )}{{\sum }_{{r}^{′}} p ( φ , ψ \vert {r}^{′} ) p ( {r}^{′} )}$$

In two dimensions, the adaptive kernel density estimate of φ, ψ for rotamer r is:

$$p ( φ , ψ \vert r ) = \frac{1}{4 {π}^{2} {N}_{r}} {\sum }_{i = 1}^{{N}_{r}} \frac{1}{{( {I}_{0} ( κ / {λ}_{i} ) )}^{2}} exp ( \frac{κ}{{λ}_{i}} ( cos ( {φ}_{i} - φ ) + cos ( {ψ}_{i} - ψ ) ) )$$

where λᵢ is dependent on φ, ψ but independent of the rotamer:

$${λ}_{i} = {( \frac{{( {\prod }_{j = 1 , N} f ( {φ}_{j} , {ψ}_{j} ) )}^{\frac{1}{N}}}{f ( {φ}_{i} , {ψ}_{i} )} )}^{\frac{1}{2}}$$

N is the total number of residues for this residue type and Nᵣ is the count for rotamer r. κ is the geometric mean of all the kernel widths. The pilot estimate f comes from a non-adaptive kernel estimate, using the same kernel width κ:

$$\hat{f} ( {φ}_{i} , {ψ}_{i} ) = \frac{1}{{N ( 2 π {I}_{0} ( κ ) )}^{2}} {\sum }_{j = 1}^{N} exp ( κ ( cos ( {φ}_{j} - {φ}_{i} ) + cos ( {ψ}_{j} - {ψ}_{i} ) ) )$$

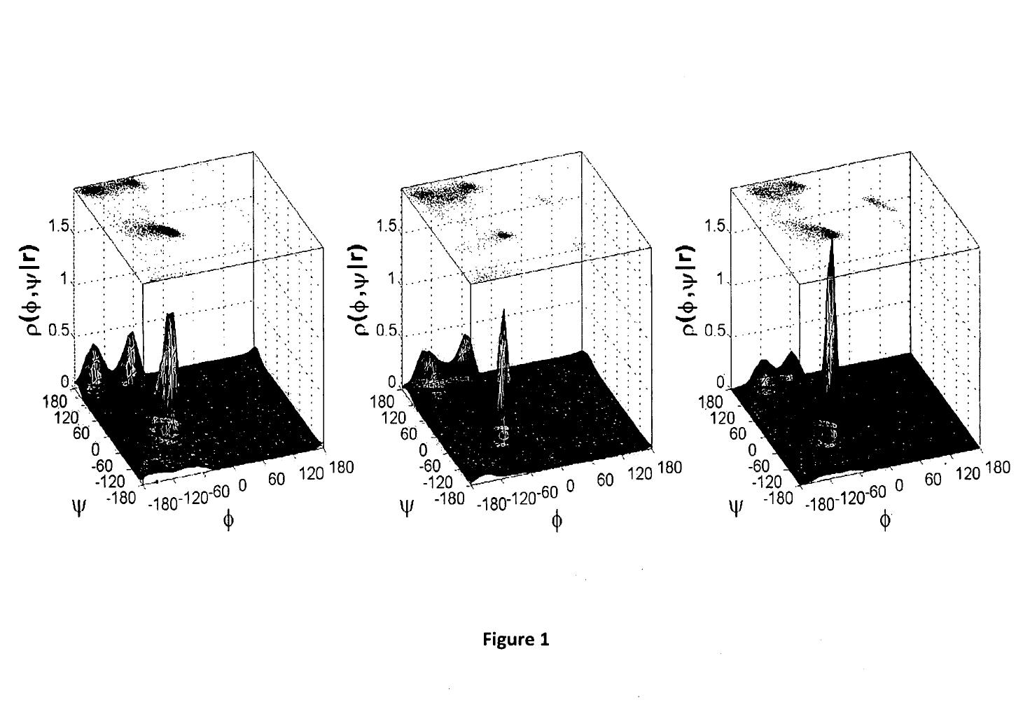

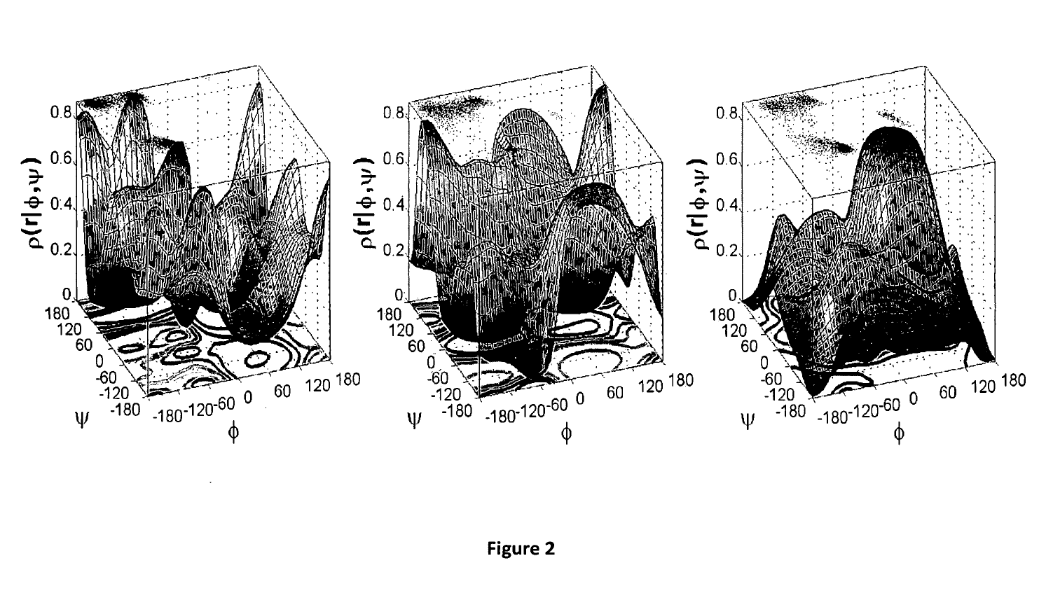

The kernel width geometric mean κ controls the extent of smoothing of the rotamer probabilities. It can be selected by penalized maximum likelihood cross-validation, such that a gamma prior probability on κ keeps κ in a range that provides the desired level of smoothness of the resulting rotamer probabilities. The functions p(φ,ψ|r) and p(r|φ,ψ) are shown in FIGS. 1 and 2, respectively.

Third, the probability distributions for the rare rotamers of long side chains can be improved by using a Bayesian prior. This can be achieved by combining kernel density estimates for the χ₁ rotamer, r₁, dependent on φ and ψ with backbone-independent estimates of the conditional probabilities of the degrees of freedom further from the backbone (χ₂, χ₃, χ₄):

\hat{f}(r|φ,ψ)∝p(r₁|φ,ψ)p(r₂|r₁)p(r₃|r₂)p(r₄|r₃)

Fourth, a significant feature of the instant rotamer libraries is the smooth φ,ψ-dependence of the means and variances of the dihedral angles for each rotamer. Due to interactions with the local backbone, both steric and electrostatic, these average angles have strong and systematic variation with φ and ψ for each rotamer. For this purpose in the present rotamer libraries, non-parametric kernel regression estimates may be used to determine the means of each χ angle as smoothly varying functions of the backbone dihedrals. For any side chain data point i, of rotamer r, and dihedral angle χ:

χᵢ=m(φᵢ,ψᵢ)+v^{1/2}(φᵢ,ψᵢ)εᵢ

where m is the regression function being sought, and ε are random errors:

m(x,y)=E(χ|φ=x,ψ=y)

v(x,y)=Var(χ|φ=x,ψ=y)

Estimates for m and v are derived from zeroth-order or Nadaraya-Watson kernel regression (Nadaraya (1964) Theory Prob. Appl., 9:141-142):

$$\hat{m} ( φ , ψ ) = \frac{{\sum }_{i = 1 , {N}_{r}} K ( {φ}_{i} - φ , {ψ}_{i} - ψ ) {χ}_{i}}{{\sum }_{i = 1 , {N}_{r}} K ( {φ}_{i} - φ , {ψ}_{i} - ψ )}$$

$$\hat{v} ( φ , ψ ) = \frac{{\sum }_{i = 1 , {N}_{r}} K ( {φ}_{i} - φ , {ψ}_{i} - ψ ) {( {χ}_{i} - \hat{m} ( {φ}_{i} , ψ ) )}^{2}}{{\sum }_{i = 1 , {N}_{r}} K ( {φ}_{i} - φ , {ψ}_{i} - ψ )}$$

For the kernel regressions, kernels that are adaptive to the density at the query point rather than each data point are used:

$$K ( φ - {φ}_{i} , ψ - {ψ}_{i} ) = \frac{1}{4 {π}^{2}} \frac{1}{{( {I}_{0} ( κ / {λ}_{φ ψ} ) )}^{2}} exp ( \frac{κ}{{λ}_{φψ}} ( cos ( {φ}_{i} - φ ) + cos ( {ψ}_{i} - ψ ) ) )$$

A scheme to adapt the kernel to the query point density based on the maximum of the pilot density over all φ,ψ can be used.

Fifth, in earlier libraries, all dihedral angle degrees of freedom were treated as “rotameric.” That is, the entire dihedral angle space was broken up into bins and conformations counted. For asparagine, for instance, in Dunbrack and Cohen (Protein Sci. (1997) 6:1661-1681), χ₂ was divided into three bins, (−90,−30], (−30,+30], (+30,+90], by considering OD1 and ND2 atoms as indistinguishable. In Dunbrack (Curr. Opin. Struct. Biol. (2002) 12:431-440), the reduce program of Word et al. (Word et al. (1999) J. Mol. Biol., 285:1735-1747) was used to place OD1 and ND2 of Asn as well as possible considering hydrogen bonding patterns. χ₂ was then divided in the range (−180,180) into six bins, with different offsets depending on the χ₁ rotamer. In each of these bins, mean dihedral angles and standard deviations were calculated. This is a poor model for the density, which is single-moded and broadly distributed, with some skewness, depending on the χ₁ rotamer. The same is true for Asp χ₂, Gln χ₃, and Glu χ₃. It is also true for Phe and Tyr χ₂, except perhaps for a minor population of a second χ₂ rotamer (mean angle ˜0°) for Phe and Tyr, when r₁=g⁻. His and Trp have two and three broad, asymmetric peaks respectively in their χ₂, densities, and may also be modeled using the same method as other non-rotameric degrees of freedom.

For the non-rotameric degrees of freedom, a density estimate over the full range of the side-chain dihedral angle for each φ,ψ on a 10°×10° grid is desired. This is effectively a regression of the χ₂ or χ₃ densities on φ and ψ. So using a novel combination of query- and data-adaptive kernel density estimates, the desired probability distribution is:

$$p ( χ \vert φ , ψ , r ) = \frac{{\sum }_{i = 1 , {N}_{r}} K ( {φ}_{i} - φ , {ψ}_{i} - ψ ) p ( χ \vert r , {χ}_{i} )}{{\sum }_{i = 1 , {N}_{r}} K ( {φ}_{i} - φ , {ψ}_{i} - ψ )}$$

$$where$$

$$K ( φ - {φ}_{i} , ψ - {ψ}_{i} ) = \frac{1}{4 {π}^{2}} \frac{1}{{( {I}_{0} ( κ / {λ}_{φ ψ} ) )}^{2}} exp ( \frac{κ}{{λ}_{φ ψ}} ( cos ( {φ}_{i} - φ ) + cos ( {ψ}_{i} - ψ ) ) )$$

$$and$$

$$p ( χ \vert r , {χ}_{i} ) = \frac{1}{2 π {N}_{r} {I}_{0} ( κ / {λ}_{i} )} exp ( \frac{κ}{{λ}_{i}} cos ( {χ}_{i} - χ ) )$$

$${λ}_{i} = {( \frac{{( {\prod }_{j = 1 , {N}_{r}} {f}_{r} ( {χ}_{j} ) )}^{\frac{1}{{N}_{r}}}}{{f}_{r} ( {χ}_{i} )} )}^{\frac{1}{2}}$$

Sixth and last, to derive average dihedral angles for rotamer libraries, one uses the angles measured from the deposited coordinates in the PDB. However, when using these rotamer libraries in structure prediction as in SCWRL or Rosetta, “standard” bond lengths and bond angles are used. These almost always come from the values of Engh and Huber, derived from peptide crystal structures (Engh and Huber (1991) Acta. Cryst., A47:392-400). A very high-resolution set of proteins (resolution ≦1.1 Å, 177 chains at 50% identity) may be used to recalculate these bond length and bond angle averages. Second, dihedral angles may be recalculated by fitting side chains coordinates in PDB entries using the fixed bond lengths and angles. This is a straightforward steepest-descent minimization of the RMSD as a function of the dihedral angles. Dihedral angles derived in this way may have different distributions than those measured directly from the coordinates. In combination with fixed bond lengths and angles used in prediction programs, these distributions will lead to more consistent and accurate structure prediction.

### Rotamer Libraries Comprising Modified Amino Acids

For modifications with sufficient data in the PDB, rotamer libraries may be developed that can be used in prediction methods similar to the ways in which standard amino acid rotamer libraries have been developed and utilized. Such modifications include but are not limited to phosphorylated serine, threonine, and tyrosine and N-linked and O-linked glycosylated asparagine and serine respectively. Most modifications change the amino acid at a position in the side chain that is further away from the backbone than the γ heavy atom. This means that there is unlikely to be a strong backbone-dependent effect on the rotamer populations of the modified part of the side chain. Therefore, conditional backbone-independent rotamer libraries may be derived, defined as p(r₂, r₃, . . . |r₁). For prediction purposes within SCWRL or other programs, these conditional probabilities can be combined with backbone-dependent probabilities for r₁ as described above for longer side chains.

p(r₁,r₂,r₃, . . . |φ,ψ)∝p(r₂,r₃, . . . )p(r₁|φ,ψ)

As with the standard amino acids, some angles may be distributed narrowly at one mode or with modes well separated compared to their variances, similar to the χ₁ dihedral of the standard amino acids. These can be modeled with the methods described above for rotameric amino acid side chains. Dihedral angles that have more complex densities can be modeled with the methods described above for non-rotameric amino acid side chains.

Other targets may include organic molecules that are present in many protein structures, even if they are not covalently bonded to the protein (e.g., ATP and S-adenosyl methionine).

### SCWRL4: A Program for Protein Side-Chain Prediction

The instant invention may be used for the prediction of protein side-chain conformations. Determination of side-chain conformations is an important step in protein structure prediction and protein design. Many such methods have been presented, although only a small number are in widespread use. SCWRL is one such method, and the SCWRL3 program (Canutescu and Dunbrack (2003) Protein Sci., 12:963-972) has remained popular due to its speed, accuracy, and ease-of-use for the purpose of homology modeling. However, higher accuracy at comparable speed is desirable. This has been achieved in SCWRL4 (see, e.g., Example 2; Krivov et al. (Proteins: Structure, Function, and Bioinformatics (2009) 77:778-795); and dunbrack.fccc.edu/scwrl) through: 1) a new backbone-dependent rotamer library based on kernel density estimates, as defined by the instant invention; 2) averaging over samples of conformations about the positions in the rotamer library; 3) a fast anisotropic hydrogen bonding function; 4) a short-range, soft van der Waals atom-atom interaction potential; 5) fast collision detection using k-discrete oriented polytopes; 6) a tree decomposition algorithm to solve the combinatorial problem; and 7) optimization of all parameters by determining the interaction graph within the crystal environment using symmetry operators of the crystallographic space group. Programs for protein side-chain prediction of the instant invention may comprise at least one, at least two, at least three, at least four, at least five, at least six, or all of the above listed characteristics. Accuracies as a function of electron density of the side chains demonstrate that side chains with higher electron density are easier to predict than those with low electron density and presumed conformational disorder. For a testing set of 379 proteins, 86% of χ₁ angles and 75% of χ₁₊₂ are predicted correctly within 40° of the X-ray positions. Excluding the lowest density side chains (<25^{th} percentile), these numbers rise to 89% and 79%. The new program may maintain its simple command-line interface, designed for homology modeling, and is available as a dynamic-linked library for incorporation into other software programs.

SCWRL4 has been used to accurately predict protein structure. For example, Friedrich et al. (Mol. Neurobiol. (2010) 41:42-51) used SCWRL4 to model the double C2 domain (DOC2). Max et al. (Proteins: Structure, Function, Bioinformatics (2009) 78:559-574) used SCWRL4 to model side chains of amino acids in β-sheets.

### MolIDE2: An Integrated Database and Application for Modeling of Proteins and Protein Complexes

While other modeling programs can be used, a graphical platform incorporating several algorithms, for modeling of single proteins based on multiple templates, and for modeling complexes is provided (termed MolIDE2) in accordance with one aspect of the instant invention. A large percentage of proteins act biologically as homodimers, heterodimers or larger oligomeric structures. To understand mechanisms, phenotypes of mutations, and other biological phenomena through protein structure prediction, it is important to be able to model the correct oligomeric state of a protein. MolIDE2 has been developed to provide homology modeling of proteins in functionally relevant states. This modeling is based on finding templates in the PDB with the desired oligomeric states or other protein-protein or protein-ligand interactions. MolIDE2 takes one or more input sequences, and first identifies the domain families that exist in the query sequence(s). It then finds all PDB templates with the same domain content from a precompiled relational database. This database, developed and distributed with MolIDE2, provides comprehensive sequence, structural, functional, and other biological information, including putative biological assemblies derived from X-ray crystal structures by the PISA database server (Krissinel and Henrick (2007) J. Mol. Biol., 372:774-797). The user may then select the template with the desired biologically relevant structure (e.g., homodimer or heterotetramer). With the selected template, MolIDE2 performs the standard steps in homology modeling, such as backbone generation, side-chain replacement, and loop modeling. The final output is a file in PDB format containing the coordinate information of the modeled protein complex.

MolIDE2 may include some or, preferably, all of the following: 1) BLAST, PSI_BLAST, HMM-HMM and profile-profile alignment methods to search for homologous templates in the PDB; 2) once one or more homologous PDB proteins are found, use of PFAM (Bateman et al. (2004) Nucleic Acids Res., 32 Database issue: D138-141) and SCOP (Murzin et al. (1995) J. Mol. Biol., 247:536-540) to identify all or nearly all proteins in the PDB homologous to the target(s); 3) multiple structure alignment of all homologous templates or a manually selected subset; 4) building of a hidden Markov model based on the multiple structure alignment with the addition of easily aligned sequences from Uniprot/PFAM; 5) sequence alignment of the query (or query profile) to the multiple structure alignment using hidden Markov model (HMM) methods; 6) identification of ligands in any of the homologous templates that may be of biological interest, and positioning of selected ligand(s) within the template multiple structure alignment; 7) building of a set of models by manual and automated selection of components from the multiple structure alignment, including a core structure, loops, and ligands, all from one or more templates; 8) modeling of both standard amino acids and their modifications with SCWRL4; 9) refinement and model completion (e.g., building of segments for which no template exists) using Rosetta or other programs; 10) tabulation of available biological units of all templates that contain one or more domains homologous to the query sequence(s); 11) building of homooligomeric models based on these biological units; and 12) building of heterooligomeric models based on these biological units.

### Rationally-Designed, Engineered Molecular Targeting Agents

Antibodies have demonstrated significant efficacy and market value in the treatment of cancer and other diseases. Antibodies that have the potential for the greatest clinical impact are often those that directly mediate a biological effect either by initiating a signaling event (e.g., the initiation of apoptosis via anti-TRAIL antibodies) or by blocking ligand binding and inhibiting signaling (e.g., the inhibition of the binding of EGF to EGFR). However, as ligand-binding sites can exhibit a high degree of conservation between species, it is often difficult using current technologies to generate functional antibodies that bind to these sites and initiate the desired biological effect. To overcome this issue, the protein structure prediction methods of the instant invention may be used to model the interactions between ligands and receptors or antigens and antibodies that play a critical role in cancer and other diseases. Using computational protein design, ligands can be engineered comprising modified functional domains which exhibit enhanced specificity and/or affinity into existing antibody frameworks. The improved signaling ligands will have clinical utility for modulating therapeutically desirable signaling events.

In accordance with another aspect of the instant invention, methods for generating an optimized structure of at least one polypeptide are provided. The polypeptide may be optimized in any characteristic desired by the user (e.g., modulated enzymatic activity, modulated binding affinity, etc.). Typically, the polypeptide will be modified to modulate the interaction (e.g., increase or decrease the affinity/avidity) for at least one other compound (e.g., a ligand, cofactor, or polypeptide). In a particular embodiment, the methods comprise the steps of (a) providing the backbone structure of the at least one polypeptide; (b) utilizing the rotamer library of the instant invention to establish a group of potential rotamers for at least one variable residue position in the polypeptide; and (c) analyzing the interaction of each of the rotamers obtained in step b) with at least part or all of the remainder of the polypeptide structure, thereby generating an optimized structure of the side chains of at least one amino acid of at least one polypeptide. In a particular embodiment, the method comprises the coordinates of a ligand and/or cofactor, which can be a polypeptide or non-proteinaceous (e.g., metal ions, organic cofactors, iron-sulfur complexes, etc.), in step (a) and the resultant optimized structure of the polypeptide is in the presence of the ligand/cofactor. In yet another embodiment, at least one variable residue position has rotamers from at least two different amino acid side chains. The methods may be employed to determine the optimal amino acid sequence of the polypeptide for binding (particularly increased binding) at least one ligand, cofactor, and/or second polypeptide. In certain embodiments, the second polypeptide may be the same as the first polypeptide (mutated or wild-type). This allows, for example, for the modulation of the interactions of dimers (e.g., homodimers) and other multimers. The methods may be performed with the assistance of a computer and the results outputted to the user in any desired manner (e.g., a print out or a display) and/or stored in memory. In yet another embodiment, the backbone structure may be determined experimentally.

In still another embodiment, the optimized polypeptide may be tested experimentally for the desired characteristic. For example, the optimized polypeptide may be tested for modulated binding of the ligand, cofactor, or second polypeptide compared to wild-type or the polypeptide prior to mutation. Binding assays are well-known in the art. In one embodiment, the polypeptide or the binding partner is immobilized. Examples of binding assays include, without limitation, surface plasmon resonance (SPR), ELISA, immunoblots, immunohistochemistry, affinity chromatography (e.g., immobilized metal affinity chromatography), size exclusion chromatography, and mass spectroscopy.

Methods of synthesizing the polypeptide are well known in the art. For example, the polypeptide may be made synthetically or recombinantly. The polypeptide may be produced using in vitro expression methods and cell-free expression systems known in the art. In vitro transcription and translation systems are commercially available, e.g., from Promega Biotech (Madison, Wis.) or Gibco-BRL (Gaithersburg, Md.). The polypeptide may be produced by expression in any suitable prokaryotic or eukaryotic system. For example, part or all of a DNA molecule encoding for the polypeptide may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences.

In a particular embodiment, the polypeptide is assayed in vivo (e.g., by expressing a nucleic acid in an animal model). In another embodiment, the polypeptide produced by gene expression in a recombinant prokaryotic or eukaryotic system may be purified according to methods known in the art. A commercially available expression/secretion system can be used, whereby the recombinant polypeptide is expressed and thereafter secreted from the host cell, and readily purified from the surrounding medium. If expression/secretion vectors are not used, an alternative approach involves purifying the recombinant polypeptide by affinity separation, such as by immunological interaction with antibodies that bind specifically to the recombinant protein or other columns (e.g., Ni columns) for isolation of recombinant polypeptides tagged with at least one purification tag (e.g., 6-8 histidine residue tags). Polypeptides of the invention, prepared by the aforementioned methods, may be analyzed according to standard procedures. For example, such protein may be subjected to amino acid sequence analysis, according to known methods.

As stated hereinabove, methods for determining whether an alteration in a first polypeptide modulates the binding of the first polypeptide to a second compound or polypeptide are provided. For example, the methods may be used to modulate the interaction of a binding pair, including, e.g., dimer interactions, receptor-ligand, antibody-antigen, receptor-hormone, receptor-ligand, agonist-antagonist, lectin-carbohydrate, etc. In a particular embodiment, the methods comprise (a) providing a set of structure coordinates for the amino acid residues of the first and second polypeptides; (b) modeling the interaction between the first and second polypeptides; (c) utilizing the rotamer library of the instant invention to establish a group of potential rotamers for at least one variable residue position in the first polypeptide, thereby producing a set of structure coordinates that define an altered first polypeptide; (d) modeling the interactions of the altered first polypeptide with the second polypeptide; and (e) determining whether the alteration modulates the interaction between the altered first polypeptide and said second polypeptide compared to the first polypeptide and the second polypeptide. In certain embodiment, the modulation of the interaction is a modulation (e.g., increase) in the avidity or binding affinity of the polypeptides. In a particular embodiment, one of the polypeptides is an antibody and the other protein is an antigen or epitope, particularly one recognized by the antibody. The methods may be performed with the assistance of a computer and the results outputted to the user in any desired manner (e.g., a print out or a display) and/or stored in memory). In yet another embodiment, the structure coordinates may be determined experimentally. In still another embodiment, and as described above, the altered first polypeptide is tested experimentally for modulated binding of the second polypeptide compared to the first polypeptide and/or wild-type.

An “antibody” or “antibody molecule” is any immunoglobulin, including antibodies and fragments thereof, that binds to a specific antigen. The term includes polyclonal, monoclonal, chimeric, single domain (Dab), minibody, diabody, tetrabody, and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)2, Fv, scFv, scFv2, scFv-Fc, single variable domain (e.g., variable heavy domain, variable light domain). Dabs can be composed of a single variable light or heavy chain domain. In a certain embodiment of the invention, the variable light domain and/or variable heavy domain modeled for the polypeptide are inserted into the backbone of the above mentioned antibody constructs. Methods for recombinantly producing antibodies are well-known in the art. For example, commercial vectors comprising constant genes to make IgGs from scFvs are provided by Lonza Biologics (Slough, United Kingdom).

The following examples are provided to illustrate various embodiments of the present invention. The examples are illustrative and are not intended to limit the invention in any way.

### Example 1

### Modeling of the CBS Bateman Domains

As a real modeling example to demonstrate some of the functionalities of MolIDE2, the C-terminal domain of human cystathionine (3-synthase (CBS) was use as a target sequence. CBS catalyzes the covalent linkage of serine and the methionine metabolite homocysteine to produce cystathionine. Cystathionine is subsequently converted into cysteine. High levels of homocysteine are strongly linked to heart disease (Refsum et al. (1998) Annu. Rev. Med., 49:31-62), and patients with homocystinuria have been found to have mutations in the gene that codes for CBS (Kraus et al. (1999) Hum. Mutat., 13:362-375). Most such patients are responsive to vitamin B6 (PLP), which is covalently linked to CBS. The enzyme domain of CBS was previously modeled based on the tryptophan synthase β chain and the positions of B6-unresponsive mutations were identified near the bound PLP.

The enzyme domain of human CBS is preceded by a 75 amino acid proline-rich region that binds heme and is followed by a 155 amino acid region C-terminal domain. This domain contains two copies of a ˜60 amino acid motif found in many proteins, including inosine monophosphate dehydrogenase, chloride channels, and the 5′-AMP-activated protein kinase. This motif is referred to as a “CBS domain”, in reference to its presence in CBS. CBS domains always appear in tandem pairs, now often referred to together as a “Bateman domain” (Bateman, A. (1997) Trends Biochem. Sci., 22:12-13). Indeed, CBS itself was long thought to have a single CBS domain, but sequence motifs were identified that indicate the presence of a second CBS domain in CBS (Shan et al. (2001) Hum. Mol. Genet., 10:635-643). In many proteins, the function of the Bateman domain appears to be to bind adenosine nucleotides, AMP and ATP, thereby modulating the activity of the associated protein. CBS is activated by the binding of S-adenosyl methionine (SAM). Elimination of the Bateman domain region produces an enzyme that is constitutively active and unresponsive to SAM (Shan and Kruger (1998) Nat. Genet., 19:91-93).

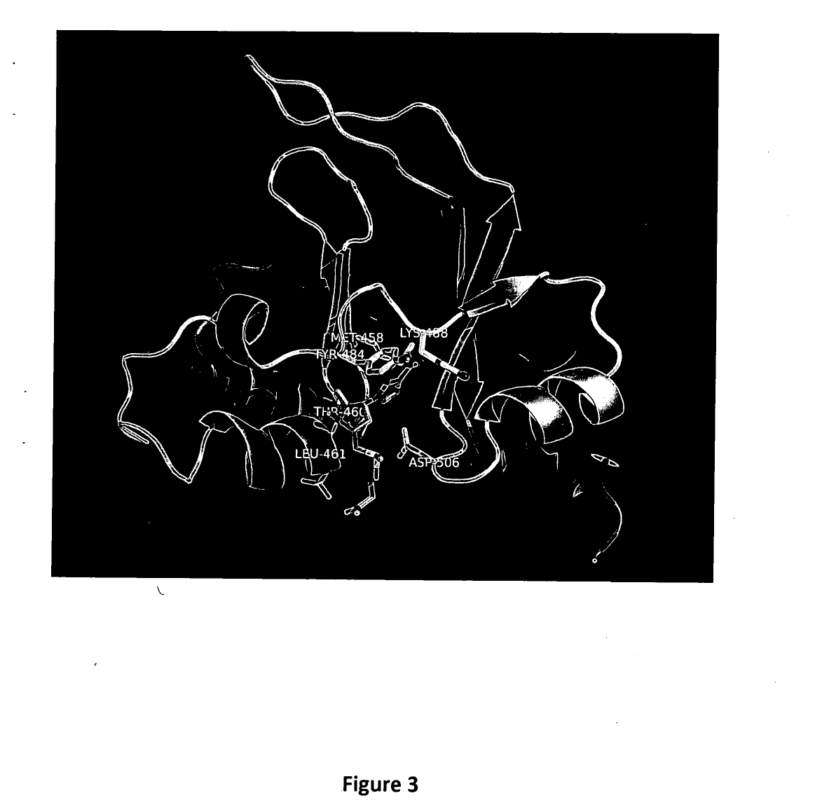

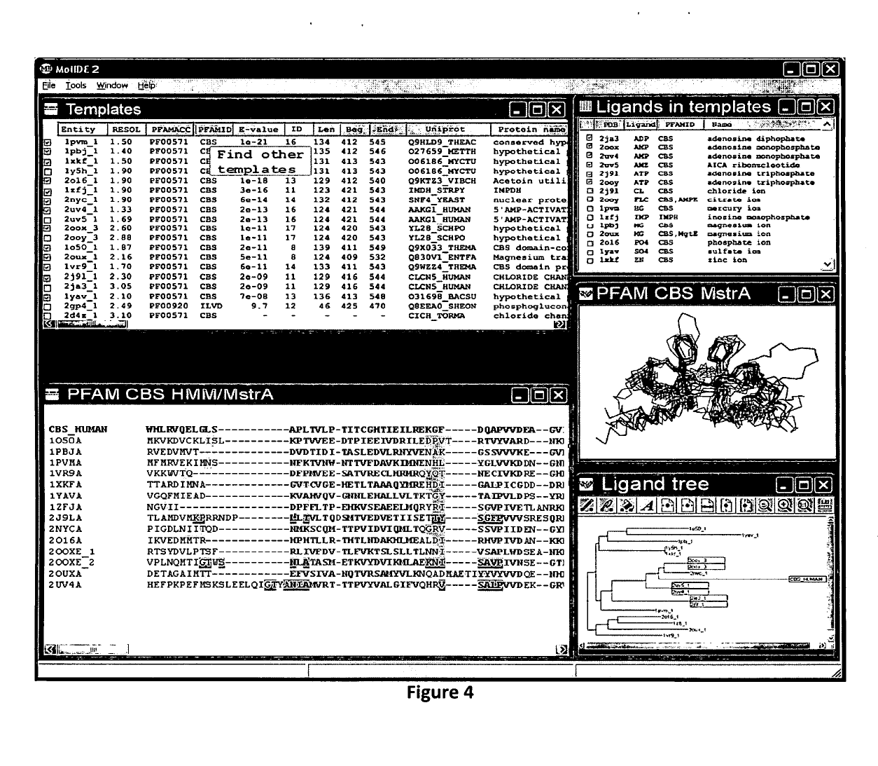

There are many questions that may be investigated with a model of the Bateman domain of CBS. First, how SAM binds to the CBS-BD and how this leads to activation of CBS can be determined. Second, the reason why some mutations in the enzyme domain affect CBS function in the full-length protein, but not in truncated proteins missing the C-terminal region can be determined. Third, it can be determined how some mutations in the CBS-BD associated with homocystinuria affect SAM activation and CBS function. Fourth, it can be determined how the Bateman domain region stabilizes the formation of homotetramers from homodimers of CBS. A model of CBS-BD is shown in FIG. 3 and FIG. 4 provides a MOLIDE2 screenshot. The model was built from PDB entries 1PVM and 2YZQ which contains bound S-adenosyl methionine. The model was built using MolIDE2 and SCWRL4, which uses the instant invention to sample and score protein side-chain conformations.

### Example 2

### SCWRL4

### Introduction

The side-chain conformation prediction problem is an integral component of protein structure determination, protein structure prediction, and protein design. In single-site mutants and in closely related proteins, the backbone often changes little and structure prediction can be accomplished by accurate side-chain prediction (Veenstra et al. (1997) Protein Eng., 10:789-807). In docking of ligands and other proteins, taking into account changes in side-chain conformation is often critical to accurate structure predictions of complexes (Gray et al. (2003) J. Mol. Biol., 331:281-299; Meiler et al. (2006) Proteins, 65:538-548; Leach, A. R. (1994) J. Mol. Biol., 235:345-356). Even in methods that take account of changes in backbone conformation, one step in the process is recalculation of side-chain conformation or “repacking” (Rohl et al. (2004) Proteins, 55:656-677). Because many backbone conformations may be sampled in model refinements, side-chain prediction must also be very fast. In protein design, as changes in the sequence are proposed by Monte Carlo steps or other algorithms, conformations of side chains need to be predicted accurately in order to determine whether the change is favorable or not (Jones, D. T. (1994) Prot. Eng., 3:567-574; Dahiyat et al. (1996) Prot. Sci., 5:895-903; Kuhlman et al. (2003) Science 302:1364-1368).

Most side-chain prediction methods are based on a sample space that depends on a rotamer library, which is a statistical clustering of observed side-chain conformations in known structures (Dunbrack, R. L (2002) Curr. Opin. Struct. Biol., 12:431-440). Such rotamer libraries can be backbone-independent, lumping all side chains together regardless of the local backbone conformation (Lovell et al. (2000) Proteins 40:389-408), or backbone-dependent, such that frequencies and dihedral angles vary with the backbone dihedral angles φ and ψ (Dunbrack et al. (1993) J. Mol. Biol., 230:543-574; Dunbrack et al. (1997) Protein Sci., 6:1661-1681). An alternative to using statistical rotamer libraries is to use conformer libraries, which are samples of side chains from known structures, usually in the form of Cartesian coordinates, thus accounting for bond length, bond angle, and dihedral angle variability (De Maeyer et al. (1997) Fold Des., 2:53-66; Shetty et al. (2003) Protein Eng., 16:963-969; Peterson et al. (2004) Protein Sci., 13:735-751; Xiang et al. (2001) J. Mol. Biol., 311:421-430). Once a search space in the form of rotamers (and samples around rotamers in some cases) or conformers is defined, a scoring function is required to evaluate the suitability of the sampled conformations. These may include the natural logarithm of the observed rotamer library frequencies (Bower et al. (1997) J. Mol. Biol., 267:1268-1282; Liang et al. (2002) Protein Sci., 11:322-331; Rohl et al. (2004) Methods Enzymol., 383:66-93; Lu et al. (2008) Protein Sci., 17:1576-1585), van der Waals or hard sphere steric interactions between atoms of different side chains, and sometimes electrostatic, hydrogen bonding, and solvation terms (Lu et al. (2008) Protein Sci., 17:1576-1585; Jackson et al. (1998) J. Mol. Biol., 276:265-285; Mendes et al. (2001) J. Comp. Aided Mol. Design, 15:721-740; Jacobson et al. (2002) J. Mol. Biol., 320:597-608; Camacho, C. J. (2005) Proteins 60:245-251). Many search algorithms have been applied, including cyclic optimization of single residues or pairs of residues (Dunbrack et al. (1993) J. Mol. Biol., 230:543-574; Xiang et al. (2001) J. Mol. Biol., 311:421-430), Monte Carlo (Rohl et al. (2004) Proteins 55:656-677; Liang et al. (2002) Protein Sci., 11:322-331; Holm et al. (1992) J. Mol. Biol., 225:93-105), dead-end elimination (Desmet et al. (1992) Nature 356:539-542; Goldstein, R. F. (1994) Biophys. J., 66:1335-1340), self-consistent mean field optimization (Koehl et al. (1994) J. Mol. Biol., 239:249-275), integer programming (Kingsford et al. (2005) Bioinformatics 21:1028-1036), and graph decomposition (Bower et al. (1997) J. Mol. Biol., 267:1268-1282; Canutescu et al. (2003) Protein Sci., 12:2001-2014; Xu, J. (2005) 9th Annual International Conference on Research in Computational Molecular Biology (RECOMB), pages 423-439). These methods vary in how fast they can solve the combinatorial problem, and whether they guarantee a global minimum of the given energy function or instead search for a low energy without such a guarantee. In general, such a guarantee is not necessary, given the approximate nature of the energy functions, and it is the overall prediction accuracy and speed that are more important features of a prediction method.

In recent years, it has become clear that some flexibility around rotameric positions (Peterson et al. (2004) Protein Sci., 13:735-751; Xiang et al. (2001) J. Mol. Biol., 311:421-430; Mendes et al. (1999) Proteins, 37:530-543) and more sophisticated energy functions (Lu et al. (2008) Protein Sci., 17:1576-1585; Jacobson et al. (2002) J. Phys. Chem. B, 106:11673-11680) are needed for improved side-chain packing and prediction.

SCWRL3 is one of the most widely used programs of its type with 2891 licenses in 72 countries as of Feb. 16, 2009. It uses a backbone-dependent rotamer library (Dunbrack et al. (1997) Protein Sci., 6:1661-1681), a simple energy function based on the library rotamer frequencies and a purely repulsive steric energy term, and a graph decomposition to solve the combinatorial packing problem (Canutescu et al. (2003) Protein Sci., 12:2001-2014). It has a number of features that have made it widely used. The first of these is speed, which has enabled the program to be used on a number of web servers that predict protein structure from sequence-structure alignments (Jaroszewski et al. (2005) Nucleic Acids Res., 33:W284-288) and may perform many hundreds of predictions per day. The second is accuracy. At the time of its publication, it was one of the most accurate side-chain prediction methods. However, a number of other methods have appeared claiming higher accuracy (Peterson et al. (2004) Protein Sci., 13:735-751; Liang et al. (2002) Protein Sci., 11:322-331; Lu et al. (2008) Protein Sci., 17:1576-1585; Liu et al. (2003) Proteins 50:49-62), although often at much longer CPU times. The third feature of SCWRL3 is usability. The program takes input PDB coordinates, optionally a new sequence, and outputs predicted coordinates with the same residue numbering and chain identifiers as the input structure with predicted coordinates for the side chains. This feature is simple but in fact many if not most side-chain prediction programs renumber the residues of the input structure and strip the chain identifiers, making them difficult to use in homology modeling. One unfortunate feature of SCWRL3 is that the graph decomposition method used may not always result in a combinatorial optimization that can be solved quickly. In such cases, the program may go on for many hours instead of finishing in a few seconds, since it lacks any heuristic method for simplifying the problem and finishing quickly.

In developing a new generation of SCWRL, called SCWRL4, there were several goals. First, it was desirable to increase the accuracy over SCWRL3 such that SCWRL4's accuracy would be comparable or better than programs developed in the last several years. Second, it was desirable to maintain the speed advantage that SCWRL has over most similar programs. Third, it was desirable to maintain the usability of the program for homology modeling and other purposes. As part of this, it was desirable to make sure that the program always solves the structure prediction program in a reasonable time, even if the graph is not sufficiently decomposable. This is accomplished with an approximation, that while not guaranteeing a global minimum of the energy function given the rotamer search space, does complete the calculation quickly in all cases tested.

Herein, the development of the SCWRL4 program is described for prediction of protein side-chain conformations. A number of different approaches were used to accomplish the goals described above. The SCWRL energy function has been improved using a new backbone-dependent rotamer library which uses kernel density estimates and regressions to provide rotamer frequencies, dihedral angles, and variances that vary smoothly as a function of the backbone dihedral angles φ and ψ. SCWRL4 also includes a short-range, soft van der Waals interaction between atoms rather than a linear repulsive-only function used in SCWRL3, as well as an anisotropic hydrogen bond function similar to that used in Rosetta (but using a different functional form that is faster to evaluate) (Kortemme et al. (2003) J. Mol. Biol., 326:1239-1259). To account for variation of dihedral angles around the mean values given in the rotamer library, the approach of Mendes et al. (Proteins (1999) 37:530-543) was used, which samples angles around the library values and averages the energy of interaction between rotamers of different side chains over these samples, resulting in a single free-energy-like scoring function. In order to determine the interaction graph, as used in SCWRL3, a method for detecting collisions (i.e., atom-atom interactions less than some distance) was implemented using k-discrete oriented polytopes (“kDOPs”). kDOPs are three-dimensional shapes with faces perpendicular to common fixed axes, such that kDOPs around two groups of atoms can be rapidly tested for overlap (Klosowski et al. (1998) IEEE Trans Visualization Comp Graphics, 4:21-36).

In SCWRL3, a graph decomposition method was used that broke down the interaction graph of residues into biconnected components, which overlap by single residues called articulation points. In most cases, this solves the graph quickly. However, with a longer-range energy function and sampling about the rotameric dihedral angles, this is no longer true. Therefore, a version of a tree decomposition of the graphs was implemented (Xu. J. (2005) 9th Annual International Conference on Research in Computational Molecular Biology (RECOMB), pages 423-439). This is almost always successful, but in a small number of cases may still not result in an easily solvable combinatorial problem. Therefore, a heuristic projection of the pairwise energies onto self-energies within some threshold was added. This approximation of the full prediction problem always results in a solution, even if it not guaranteed to find the global minimum. Finally, the new program has been developed as a library, so that its functions can be called easily by other programs such as loop modeling and protein design.

### Methods

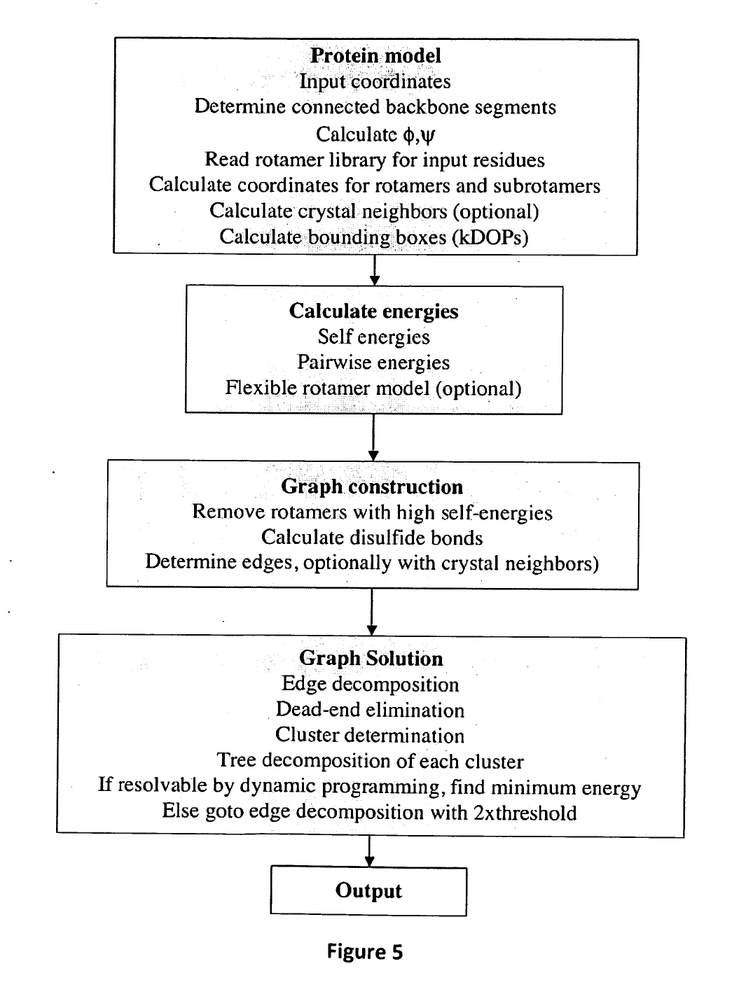

In FIG. 5, a flowchart of the basic steps in SCWRL4 to solve the side-chain prediction problem is shown. These will be discussed further below. The major steps are 1) inputting the data and constructing the side-chain coordinates; 2) calculating energies; 3) graph computation, with symmetry operators if any; 4) graph solution via edge decomposition, dead-end elimination, and tree decomposition; 5) outputting the results. SCWRL4 runs on a command line with a number of required and optional flags. A number of other options and parameters are specified in a required initialization file with extension .ini, which uses a standard name=value format (see, e.g., en.wikipedia.org/wiki/INI_file).

### Input and Construction of Coordinates.

An individual residue position is defined by specifying four backbone atoms (N,Cα, C,O) in a PDB-format input file. These individual residue sites can comprise one or more polypeptide chains, from which the backbone dihedral angles φ and ψ are calculated for each residue. For purposes of looking up residues in the rotamer library, the N-terminal residue φ is set to −60°. Similarly for C-terminal residues, ψ is set to 60°. These values are those for which there is weak dependence of the rotamer probabilities on the missing dihedral angle (Dunbrack et al. (1994) Nature Struct. Biol., 1:334-340). The Cᵢ₊₁-Nᵢ atom distances are checked to determine whether there are missing internal residues in a chain.

For each residue, rotamers are read from the backbone-dependent rotamer library of the instant invention. This rotamer library is based on a much larger data set, and is derived using kernel density estimates and kernel regressions. The rotamer library includes rotamer frequencies and mean dihedral angles and their standard deviations over a discrete (φ,ψ)-grid. This library includes much greater detail for non-rotameric degrees of freedom, in particular χ₂ for Asn, Asp, His, Phe, Trp, and Tyr and χ₃ for Glu and Gln. Optionally SCWRL4 can determine frequencies and dihedral angle parameters by bilinear interpolation from the four neighboring grid points in the library. For each χ₁ rotamer of Ser and Thr, SCWRL4 generates three rotamers for the hydroxyl hydrogen with χ₂ dihedral set to −60°, +60° and 180° and the variance set to 10°. For each χ₁. χ₂ rotamer of Tyr, two rotamers are generated for the hydroxyl hydrogen with χ₃ dihedral set to 0° and 180°, which are the values observed in neutron diffraction studies (Kossiakoff et al. (1990) Proc. Natl. Acad. Sci., 87:4468-4472). For His, extra rotamers are created for the singly protonated states (proton on ND1 or NE2). SCWRL4 does not consider charged His, since this is relatively uncommon.

Side-chain coordinates are built for all rotamers and for subrotamers about these rotamers used by the Flexible Rotamer Model (FRM, see below), typically with dihedral angles ±one standard deviation (or a fixed proportion thereof). For subrotamers, only one dihedral at a time differs from the library value, since it was found that allowing multiple deviations did not improve the accuracy but did slow the calculation. Side chains are represented in a tree-like structure, so that atoms common to more than one conformation (e.g., same CG position for different χ₂ conformers) are calculated and stored only once (Leaver-Fay et al. (2005) Algorithms in Bioinformatics, Volume 3692, Lecture Notes in Computer Science. Berlin: Springer, pages 389-400). Coordinates are built using a fast incremental torsion to Cartesian conversion method (Parsons et al. (2005) J. Comput. Chem., 26:1063-1068).

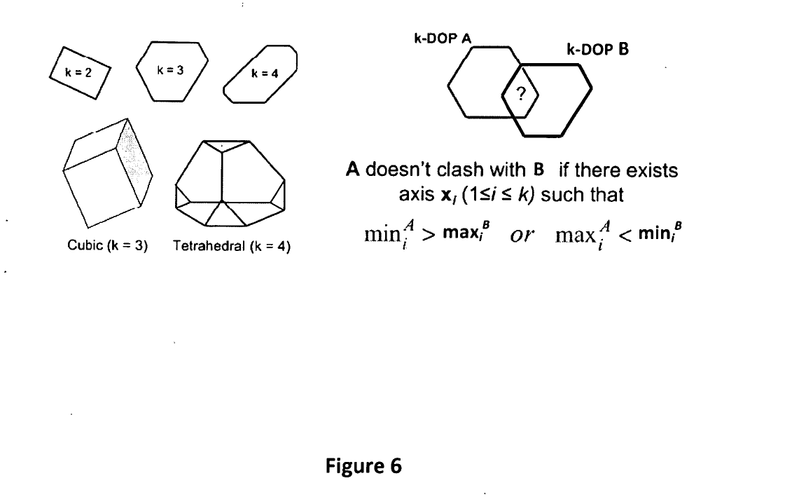

Because SCWRL4 uses a large number of rotamers and subrotamers, a fast collision detection algorithm based on k-dimensional Discrete Oriented Polytopes or kDOPs was implemented (Klosowski et al. (1998) IEEE Trans Visualization Comp Graphics, 4:21-36). The kDOPs algorithm is based on two key ideas. The first idea is to enclose each geometric object into a convex polytope of a special kind and use these as bounding boxes for clash checks. A particular class of kDOPs is defined by a set of k pairwise non-collinear unit vectors, and consists of all convex polytopes with 2k facets such that any facet is perpendicular to one of these vectors. Examples are shown in FIG. 6. For instance, if k=3, these could be the x, y, and z-axes, so that all bounding boxes are rectangular parallelepipeds whose faces are perpendicular to the Cartesian axes. The second key idea is to organize all bounding boxes related to a particular group of geometric objects as a hierarchy. These hierarchies can then be used for efficient search of all possible clashes between individual bounding boxes. The advantage of using a single set of vectors for defining the bounding boxes is that two bounding boxes of the same kDOPs class can be efficiently checked for clashes, as illustrated in FIG. 6. This is done by testing for overlaps of the real intervals that represent their projections across the corresponding basic axes. Let {aᵢ}ᵢ₌₁^{k} and {bᵢ}ᵢ₌₁^{k} be the sets of these intervals for two kDOPs “A” and “B” respectively. “A” does not clash with “B” if there exists i β{1 . . . k} such that min (aᵢ)>max (bᵢ) or if there exists j ε{1 . . . k} such that min (bⱼ)>max (aⱼ).

If neither of these conditions are met, then the underlying objects enclosed inside of these two boxes may clash, but do not necessarily do so. The last is to be checked by pairwise distance calculations of the objects enclosed in the bounding boxes.

Building a kDOP around geometric objects consists of finding projections of the objects onto the basic axes. Both the van der Waals function and the hydrogen bond potentials described in the next section have a certain boundary distance beyond which the potential is zero. These distances are used to represent each atom as a sphere with a certain radius. To build a bounding box around a whole side chain, each atom is enclosed into a kDOP and then the elementary shells are merged. The rotamers and subrotamers of a side chain can be enclosed into a single kDOP, such that all residue-residue interactions can be checked very quickly. In SCWRL4, four basic axes and construct bounding boxes around individual atoms, backbone atoms of each residue, parts of each side chain, individual rotamers (i.e. entire set of its subrotamers) and every residue (all rotamers) are use. The basic axes form a tetrahedral geometry:

$${\vec{e}}_{1 , 2} = \frac{{\vec{e}}_{z} \pm \sqrt{2} {\vec{e}}_{x}}{\sqrt{3}}$$

$${\vec{e}}_{3 , 4} = \frac{- {\vec{e}}_{x} \pm \sqrt{2} {\vec{e}}_{y}}{\sqrt{3}}$$

### Calculating of Self Energies and Pairwise Energies Via Modified Flexible Rotamer Model.

SCWRL4 uses both a rigid rotamer model (RRM), as in SCWRL3, and a flexible rotamer model (FRM) (Mendes et al. (1999) Proteins, 37:530-543). In the rigid rotamer model, the total energy of the system is expressed as:

$$E ( r ) = {\sum }_{i = 1}^{N} {E}_{self} ( {r}_{i} ) + {\sum }_{i = 1}^{N} {\sum }_{j = i + 1}^{N} {E}_{pair} ( {r}_{i} , {r}_{j} )$$

where the vector r lists a single rotamer for each of N residues in the system. In this case, the self energy of each rotamer is:

$${E}_{self} ( {r}_{i} ) = - {k}_{i} log \frac{p ( {r}_{i} )}{p ( {r}_{max} )} + {E}_{frame} ( {r}_{i} )$$

where the first term expresses the rotamer energy relative to the most populated rotamer, rmax, given the backbone dihedrals φ and ψ of residue i and the frame term expresses interaction of the side chain with the backbone and any ligand or other fixed atoms present. The value of the constant in front of the log term is allowed to be residue-dependent.

In contrast to SCWRL3, in SCWRL4 the frame and pairwise rotamer energies consist of repulsive and attractive van der Waals terms as well as a hydrogen bonding term. The repulsive van der Waals term is similar to the piecewise linear term used in SCWRL3, but is combined with a short-range attractive potential as follows. If Rᵢⱼ is the sum of the hard-sphere radii of atoms i and j and Eᵢⱼ is \sqrt{EᵢEⱼ}, where the Eᵢ values are the Eₘᵢₙ values from the CHARMM param19 potential (Brooks et al. (1983) J. Comput. Chem., 4:187-217), and d is the distance between the two atoms:

$${E}_{vdw} ( d ) = \{ \begin{matrix}10 & if \frac{d}{{R}_{ij}} \leq 0.8254 \\ 57.273 ( 1 - \frac{d}{{R}_{ij}} ) & if 0.5284 \leq \frac{d}{{R}_{ij}} \leq 1 \\ {{E}_{ij} ( 10 - 9 \frac{d}{{R}_{ij }} )}^{\frac{57.273}{9 {H}_{ij }}} - {E}_{ij} & if 1 < \frac{d}{{R}_{ij}} < \frac{10}{9} \\ {{E}_{ij} ( 9 \frac{d}{{R}_{ij}} - 10 )}^{2} - {E}_{ij} & if \frac{10}{9} \leq \frac{d}{{R}_{ij}} < \frac{4}{3} \\ 0 & if \frac{d}{{R}_{ij}} \geq \frac{4}{3} \end{matrix}$$

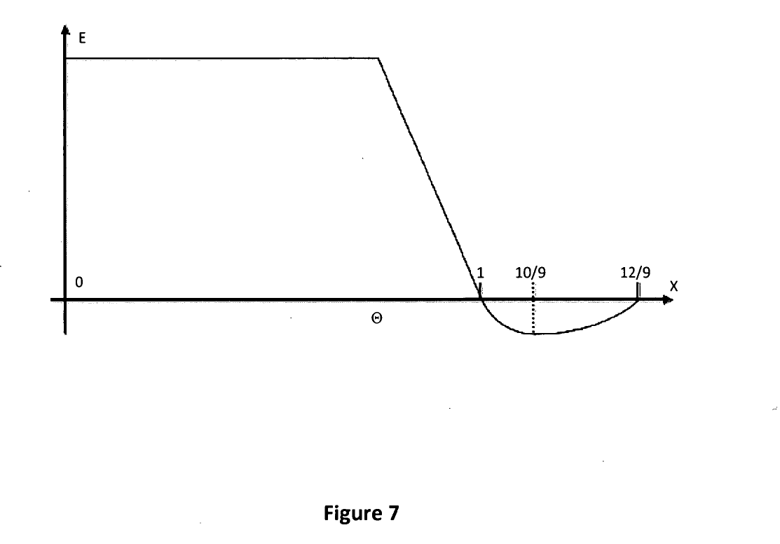

This potential is shown in FIG. 7. The hard-sphere radii were manually optimized for the training set accuracies.

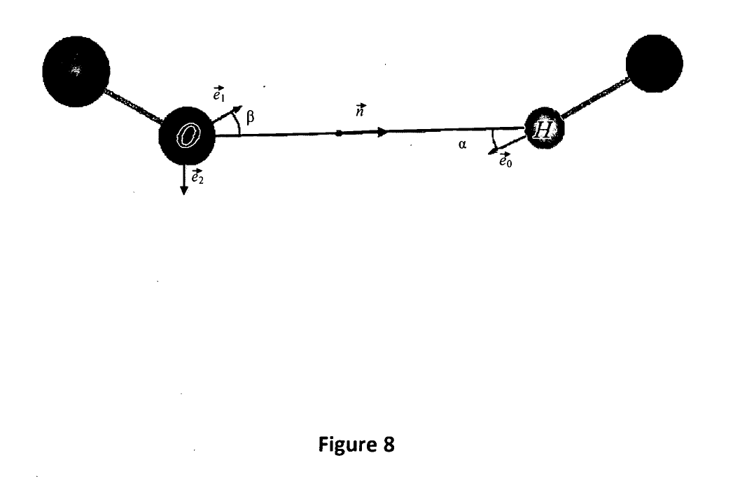

The hydrogen bonding term in SCWRL4 is similar to the one used in Rosetta (Kortemme et al. (2003) J. Mol. Biol., 326:1239-1259), although it is parameterized in a different way, as shown in FIG. 8. d is defined in this case as the distance between a polar hydrogen (HN— or HO—) and a hydrogen bond acceptor (oxygen), n as a unit vector from O acceptor to H, e₀ as a unit vector along the covalent bond from the hydrogen bond donor heavy atom (D) to H, and two unit vectors e₁ and e₂ from the hydrogen bond acceptor toward the middle of the oxygen lone-pair electron clouds. For carbonyl oxygens these two vectors are 120° apart from the double bond and coplanar with the carbonyl carbon substituents. For hydroxyl oxygens, these two vectors are 109.5° from each other and from the other two oxygen substituents (H and C), forming a tetrahedral arrangement. The hydrogen bond function is evaluated for both e₁ and e₂. For e₁, the weight w for the hydrogen bond is defined:

$$w = \frac{\sqrt{( {σ}_{d}^{2} - {( d - {d}_{0} )}^{2} ) ( cos α - cos {α}_{ma x} ) ( cos β - cos {β}_{ma x} )}}{{σ}_{d} \sqrt{( 1 - cos {α}_{ma x} ) ( 1 - cos {β}_{m ax} )}}$$

where α=cos⁻¹(− [symbol] · [symbol] ) is the angle between the D—H bond and the H . . . O vector and β=cos⁻¹( [symbol] · [symbol] ) is the angle between the O-lone pair and the O . . . H vector. If the multiplicand under the square root is negative then the score is set to zero. The calculation of this score enables an efficient implementation and together with the distance d and vector n can be done within 30 arithmetic operations, one division, and two square root evaluations.

After the weight w has been computed it is used to derive the final energy of oxygen-hydrogen interaction by balancing the default van der Waals energy and pure hydrogen-bond attraction terms:

E_{[O,H]}=(1−w)E_{vdw}+wBq_{H}qₒ

where q_{H} and q_{O} are the charges from the CHARMM param19 potential. The formulas above include five atom-independent coefficients: d₀, σ_{d}, αₘₐₓ, βₘₐₓ B. The values of these coefficients were optimized on the training set proteins.

Using single rotamers sometimes results in poor packing predictions, due to fluctuations in the dihedral angles and imprecise representations of the backbone in homology modeling. The use of subrotamers was investigated, which are defines as conformations that differ in one or more dihedral angles by one standard deviation (or some constant times this value) from the mean values given in the rotamer library:

χᵢ→{χᵢ,χᵢ−δᵢ,χᵢ+δᵢ}

If variations are allowed in all dihedral angles in this manner, treating the subrotamers as additional rotamers resulted in intractable calculations using the graph decomposition algorithm used in SCWRL3. Even with the tree decomposition of Xu (Xu, J. (2005) 9th Annual International Conference on Research in Computational Molecular Biology (RECOMB), pages 423-439), implemented in SCWRL4 (see below), the calculations often remained intractable. So the flexible rotamer model of Mendes et al. (Mendes et al. (1999) Proteins 37:530-543) was implemented, in which the subrotamers are integrated to produce an approximate free energy using the Kirkwood superposition approximation (Kirkwood, J. G. (1935) J. Chem. Phys., 3:300-313):

$$A ( r ) = {\sum }_{i = 1}^{N} {A}_{self} ( {r}_{i} ) + {\sum }_{i = 1}^{N} {\sum }_{j = i + 1}^{N} {A}_{pair} ( {r}_{i} , {r}_{j} )$$

The first term is treated as the “self free energy” and the second term as the “pairwise free energy”. A_{self} (rᵢ) and Aₚₐᵢᵣ (rᵢ, rⱼ) are defined as:

$${A}_{self} ( {r}_{i} ) = - {k}_{i} , log ( \frac{p ( {r}_{i} )}{p ( {r}_{ma x} )} ) - {T}_{i} log {\sum }_{{s}_{i} = 1}^{m} exp ( \frac{- ( {E}_{frame} ( {r}_{i} , {s}_{i} ) )}{{T}_{i}} )$$

$${A}_{pair} ( {r}_{i} , {r}_{j} ) = - {T}_{ij} log {\sum }_{{s}_{i} = 1 , n} {\sum }_{{s}_{j} = 1 , m} exp [ - \frac{{E}_{frame} ( {r}_{i} , {s}_{i} ) + {E}_{frame} ( {r}_{j} , {s}_{j} ) + {E}_{pair} ( {r}_{i} , {s}_{i} , {r}_{j} , {s}_{j} )}{{T}_{ij}} ] - {A}_{frame} ( {r}_{i} ) - {A}_{frame} ( {r}_{j} )$$

The terms E_{frame} (rᵢ, sᵢ) and A_{frame} (rᵢ) contain only the van der Waals and hydrogen bond energies. In this implementation, each residue type has a separately optimized temperature, and for the pairwise free energy, Tᵢⱼ=(Tᵢ+Tⱼ)/2.

### Graph Construction.

As with SCWRL3, some rotamers with high self-energy are removed from the calculation, since they are very unlikely to be part of the predicted structure. These rotamers are marked as inactive. In SCWRL3, rotamers with self-energy above a certain residue-independent bound were inactivated. However it sometimes happens that all rotamers have self-energy above this bound. In this case all rotamers were reactivated. In SCWRL4 this heuristic was replaced by making the bound relative to the lowest energy rotamer for each side chain. This approach guarantees that at least one rotamer will remain active. The exact value of the threshold can be customized through the configuration file.

Before the graph is constructed, disulfide bonds are resolved. SCWRL4 uses the same criterion as SCWRL3 to identify if two cysteine residues can form a disulfide bond, but introduces a new procedure for resolving ambiguities. An ambiguity occurs when more than one rotamer of a particular cysteine residue can form a disulfide bond or when one rotamer can form disulfide bonds with more than one other cysteine side chain. To select a particular collision-free combination of disulfide bonds, SCWRL4 minimizes the total energy of all disulfide bonds with respect to the mutually exclusive constraints. To do this an objective function was used in the form:

$$Φ [ η ] = {\sum }_{a} η ( a ) E ( a ) + {\sum }_{b , c} \{ \begin{matrix}C η ( b ) η ( c ) , if b and c are mutually exclusive \\ 0 , otherwise \end{matrix}$$

where the summations run over all possible disulfide bonds, C is a large positive constant and η is a binary function that evaluates to one if a particular disulfide bond is switched on and to zero otherwise. The functional above is of the same form as the one used to compute the total energy of rotamer assignment. Therefore it can be minimized it for function η via the same optimization procedure. Doing this yields a list of the optimal disulfide bonds that do not have collisions. If for a particular residue one of the rotamers participates in an optimal disulfide bond then all other rotamers are inactivated for that residue. Energies of interaction of cysteines in disulfides are added to the self-energies of rotamers of side chains within interacting distance.

SCWRL uses an interaction graph to represent the side-chain placement problem (Bower et al. (1997) J. Mol. Biol., 267:1268-1282; Canutescu et al. (2003) Protein Sci., 12:2001-2014). In this graph, vertices represent residues while edges between vertices indicate that at least one rotamer of one residue has a non-zero interaction with rotamers from another residue connected by the edge. For a single protein or protein complex, the graph is constructed by checking for overlap of the kDOP around whole residues, and then by rotamers and subrotamers. If at least one rotamer or subrotamer of one residue can interact with non-zero energy with a rotamer or subrotamer of another residue, then an edge is added to the graph between the vertices in the graph representing these residues.

SCWRL4 is able to model side chains in symmetric complexes and in the crystal using the symmetry operators of the space group given in the input PDB file CRYST1 record. For crystals, if the input PDB file contains the asymmetric unit, all residues in asymmetric units that may contact the input coordinates are constructed, as described (Xu et al. (2008) J. Mol. Biol., 381:487-507). Bounding boxes are constructed around the residues, rotamers, subrotamers, and atoms of the symmetry copies. Interactions between atoms in the input structure and its side chains and atoms in the symmetry copies and their side chains are determined. If side chains in the input structure interact with the backbone or ligands of the symmetry copies, then the static frame energies of these residues are modified accordingly. If the side chains of the input structure interact with the side chains of the symmetry copies, then an edge is created between the corresponding residues, if it does not already exist. If it does, then the pairwise energies are modified to account for the additional interactions between symmetry-related proteins. Thus a residue on one side of a protein may have an edge with a residue on the other side of a protein because of crystal symmetry.

### Graph Solution Via Tree Decomposition.

Before the major optimization via dynamic programming is launched the interaction graph undergoes some preprocessing consisting of edge decomposition and dead-end elimination. Typically this eliminates a significant number of rotamers as well as some edges and nodes. Because edges were formed based on overlapping of bounding boxes some of them may contain only zeros as pairwise rotamer-rotamer energies. If this is the case or if the actual energies of interactions are very close to zero then the edge is removed.

Edge decomposition removes edges that can be approximated as the sum over single-residue energies. If this representation is feasible within a certain threshold then the corresponding self-energies are modified and the edge is removed. With larger thresholds, more edges may be removed. In this preprocessing stage, the threshold is set to a very small value, β=0.02 kcal/mol.

The pairwise energies of two residues, Eₚₐᵢᵣ (rᵢ, rⱼ), in the rigid rotamer model or free energies, Aₚₐᵢᵣ (rᵢ, rⱼ), in the flexible rotamer model, may be represented by a matrix of real numbers eₖₗ for rotamers k=1 . . . m and l=1 . . . n. The task is to find two sets of real numbers {aₖ}ₖ₌₁^{m} and {bₗ}ₗ₌₁ⁿ which minimize the average deviation:

$$δ = {\sum }_{k = 1 , m} {\sum }_{l = 1 , n} {( {a}_{k} + {b}_{l} - {e}_{kl} )}^{2}$$

By setting the partial derivatives of δ with respect to aₖ and bₗ to zero, these two sets should satisfy the following equations:

$${a}_{k} = - \overline{b} + \frac{1}{n} {{\sum }_{l = 1}}^{n} {e}_{kl}$$

$${b}_{l} = - \overline{a} + \frac{1}{m} {\sum }_{k = 1}^{m} {e}_{kl}$$

The initial task is not well defined as its solution is not unique. Thus adding some value to all aₖ and subtracting the same value of all bₗ does not change the sum aₖ+bₗ. Therefore a can be set to an arbitrary value. For example it can be set:

$$\overline{a} = \frac{\overline{e}}{2} = \frac{1}{2 mn} {\sum }_{k = 1 , m} {\sum }_{l = 1 , n} {e}_{kl}$$

Substituting this value into the second equation, the corresponding value for b can be found:

$$\overline{b} = \frac{\overline{e}}{2}$$

Using these values, aₖ and bₗ can be determined and the maximal absolute deviation can be evaluated:

$$ɛ = {max}_{k , l} \vert {e}_{kl} - {a}_{k} - {b}_{l} \vert$$

SCWRL4 checks if this deviation is less than a certain threshold and if so then the corresponding edge can be removed and the self-energies of the kth rotamer of residue i and the lth rotamer of residue j can be modified:

E_{self}(rᵢ=k)→E_{self}(rᵢ=k)+aₖ

E_{self}(rⱼ=l)→E_{self}(rⱼ=l)+bₗ

As stated earlier, the initial value of the threshold is 0.02, which enables the algorithm to eliminate almost all redundant “near-zero” edges. Those nodes that now have zero edges can be removed from the graph; its assigned rotamer is that of lowest E_{self}.

The next step is to perform dead-end elimination (DEE) that identifies and removes rotamers that cannot be the part of the global solution. These rotamers are identified via Goldstein's criterion that was used in SCWRL3 (Goldstein, R. F. (1994) Biophys. J., 66:1335-1340). If for a certain residue only one rotamer is left after DEE then that rotamer is part of the solution. If this residue has adjacent edges then all pairwise energies with the remaining rotamer are incorporated into self-energies of the corresponding rotamers from adjacent residues and these edges are removed. This makes the residue isolated, which means that it can be removed from the graph; the self-energy of its single rotamer is added to the total value of the minimal energy. The edge decomposition and DEE steps are repeated until nothing further is removed.

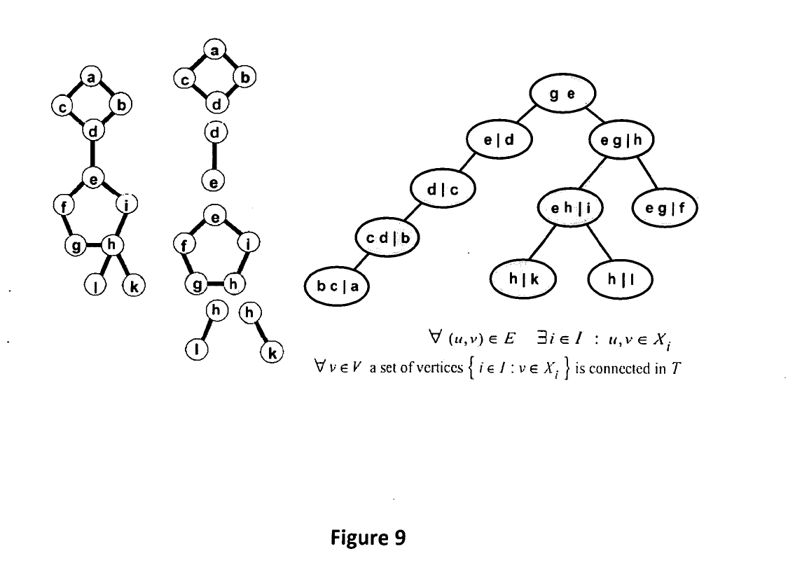

As in SCWRL3, the resulting graph may contain separated graphs or clusters with no edges between them; each of these clusters is then subject to graph decomposition. In SCWRL3, the graph decomposition was based on the determination of biconnected components, which are subgraphs that cannot be broken into parts by the removal of a single node. The graph is then a set of biconnected components connected by single nodes called articulation points. Tree decomposition can be viewed as a generalization of graph decomposition based on biconnected components (Xu, J. (2005) 9th Annual International Conference on Research in Computational Molecular Biology (RECOMB), pages 423-439). To see this, it is shown in FIG. 9 the same graph as described in the SCWRL3 paper, its decomposition into biconnected components, and its tree decomposition.

Definition: A tree-decomposition of a graph G=(V,E) is a pair (T,Z), where T=(W,F) is a tree (i.e., a graph with no cycles) with vertex set W and edge set F and Z={Z_{w}:Z_{w}⊂V}_{wεW} is a family of subsets of the set V associated one-to-one with the vertices of T that satisfies the conditions:

1. U_{wεW} Z_{w}=V

2. ∀(u,v)εE ∃w εW:u,v,εZ_{w}

3. ∀vεV a set of vertices {wεW:vεZ_{w}} is connected in T

Due to the one-to-one correspondence between sets Z and W, the vertices of tree T may be denoted as “bags”. FIG. 9 shows that condition 1 is satisfied by this tree decomposition, since all the residues are present in one or more bags. Residues c,d illustrate that condition 2 is satisfied since the edge c-d is contained in at least one bag (in this case, two). Residue h illustrates condition 3, since all the bags that contain h are connected in a single subtree.

Typically several different tree-decompositions can be built for a given graph. The width of a particular tree-decomposition is the size of the largest bag minus one. For a given graph a tree-decomposition with the minimal possible width is the optimal one and its width is called the treewidth of the graph. This characteristic indicates how well a graph is tree-decomposable. For example if a graph has no cycles (and thus is a tree) then its treewidth equals one, while for a simple cycle the treewidth equals two.

In SCWRL3, the graph solution begins with any biconnected component with a single articulation point by finding the minimum energy of the biconnected component residues for each rotamer of the articulation point. This energy is then added to the self-energy of the articulation point rotamer, and the rotamers of the biconnected component that achieve this minimum energy are assigned to the articulation point rotamer. The biconnected component can then be removed, and the process continues for all biconnected components with one articulation point in the remaining graph. The combinatorial problem is thus reduced to the order of the largest biconnected component (i.e., the one with the largest number of rotamer combinations).

In a tree decomposition, instead of using single nodes to separate the graph, the graph can be separated by removing one, two, or more nodes. To see this, in the tree decomposition in FIG. 9, each bag w is broken up into two sets of residues, Lw and Rw, where the residues in Lw are those residues in the bag that are shared between the bag and its immediate parent bag. For each bag in the figure, these are listed to the left of a vertical bar. The remaining residues in the bag, the set Rw, are those not in the parent and are placed to the right of the vertical bar. Each set Lw is a separation set of the graph G45; that is removing the residues in Lw breaks the original residue graph into two or more separate unconnected graphs. For instance, removing residues b and c breaks the original graph into two rest of the graph below residues b and c.

Solving for the minimum energy of the graph proceeds as it does in SCWRL3. Starting at a leaf (a bag with no children), y, e.g. the one containing “b c l a”, find the lowest energy of the residue(s) in Ry (in this case residue a) for each combination of the rotamers in Ly (in this case, residues b and c), saving the corresponding assignment of rotamers in Ry. Then add these energies to that rotamer combination in the parent bag, which by the definition of tree decomposition contains b and c. The procedure continues up the tree to the parent node of y (e.g., node z). Again, the minimum energy of all the rotamer combinations of those residues in Rz is calculated for each rotamer combination in set Lz of the parent. These energies need to include the energies for b,c calculated for the child node y. This procedure continues until only the root bag is left. A more formal description of this procedure is below.

The complexity of the solution is associated with the width of the tree, since all the rotamer combinations of the residues in each bag need to be enumerated. It is in general difficult to compute a treewidth and to find the optimal tree-decomposition, and it has been proved to be NP-hard for an arbitrary graph (Arnborg et al. (1987) SIAM J Algebraic and Discrete Methods, 8:277-284). For building a tree-decomposition a heuristic algorithm was developed.

In the first step, the family of sets Z is built. The input graph is gradually disassembled using a loop of the following steps:

1. Select any vertex with the minimal number of adjacent edges.

2. Form a bag of the tree from this vertex and all its neighbors. The selected vertex will be denoted as the primary vertex of the corresponding bag.

3. Add edges into the graph being processed so that the neighbors of the selected vertex become a clique (a subgraph where all nodes have edges to each other).

4. Remove the selected vertex and all adjacent edges from the graph.

5. Repeat from the first step until there are no more vertices left in the graph.

Thus a set of “bags” is obtained which represent the vertices of the tree-decomposition.

Bags are numbered in the order of their construction. It is important to notice here that within any iteration the intersection of the bag w with the vertices of the remaining graph (Sw) consists solely of the neighbors (Nw) of the initial vertex of the bag concerned, Zw∩Sw=Nw. The one-to-one correspondence between vertices and the bags verifies that the first condition in the definition of tree decomposition is automatically satisfied. Also it is clear that the edges are removed solely during the bag construction and that when any edge is removed both adjacent vertices are included into a bag. This guarantees that the second condition in the definition of a tree-decomposition is satisfied.

The second step is to connect the “bags” to obtain a tree that meets the definition of tree decomposition. This is done by sequential fastening of these bags to the tree in the reverse order in which they were constructed. Thus the bag that was created last becomes the root of the tree. The next bag becomes connected to it and thus becomes its immediate child. For the next bag, there are two choices for where to attach it. However, the appropriate node of the tree-decomposition must meet the following condition:

$$( \begin{matrix}vertices in the bag \\ to be added\end{matrix} ) ⋂ ( \begin{matrix}vertices that are \\ already on the tree\end{matrix} ) ⊆ ( \begin{matrix}vertices in the \\ appropriate bag\end{matrix} )$$

According to this condition a set of vertices in the appropriate node must contain all vertices from the bag to be added that are already present in the tree.

The tree decomposition just obtained undergoes some additional minor processing. This consists of two normalization rules, which are applied until they cannot be applied further: 1) If all vertices associated with some bag belong to the vertex set of its immediate parent then this node is removed and all its immediate children are reconnected to the parent node. 2) If some bag contains all vertices associated with its parent node then the parent bag is substituted by this bag which thus moves up one edge towards the root.

The minimum energy rotamer configuration is calculated as follows. Starting with a leaf node consisting of sets L and R, the left and right portions of each bag as defined above, define <L> as the set of all rotamer combinations of the residues in the set L, and similarly define <R> for set R. A single member of <L> denoted as l, which is a vector of rotamer assignments, one rotamer lᵢ for each residue i in the set L; similarly define r for <R>. For a leaf node, energies are calculated for each vector l:

$${ɛ}_{m i n} ( 1 ) = {min}_{r ∈ ( R )} \tilde{E} ( 1 ; r )$$

$${r}_{m i n} ( 1 ) = {argmin}_{r ∈ ( R )} \tilde{E} ( 1 ; r )$$

$$where$$

$$\tilde{E} ( 1 ; r ) = {E}_{L} ( 1 ; r ) + {E}_{R} ( r )$$

$$and$$

$${E}_{L} ( 1 ; r ) = {\sum }_{i ∈ L} {\sum }_{j ∈ R} {E}_{pair} ( {l}_{i} , {r}_{j} )$$

$${E}_{R} ( r ) = {\sum }_{j ∈ R} {E}_{self} ( {r}_{j} ) + {\sum }_{j ∈ R} {\sum }_{{k ∈ R}_{k > j}} {E}_{pair} ( {r}_{j} , {r}_{k} )$$

For fixed rotamers in L, only the pairwise interactions with rotamers in R are included, while both self and pairwise interactions among the rotamers in R must be added. For the flexible rotamer model, the values of A_{self} and Aₚₐᵢᵣ are used instead E_{self} and Eₚₐᵢᵣ. For an inner node, add in the energies assigned to rotamer combinations in the node via its children:

$$\tilde{E} ( 1 ; r ) = {E}_{L} ( 1 ; r ) + {E}_{R} ( r ) + {E}_{S} ( 1 ; r )$$

$$where$$

$${E}_{S} ( 1 ; r ) = {\sum }_{c ∈ C} ɛ ( {1}_{c} )$$

where the sum is over the children c of the inner node, and l_{c} is a vector of the rotamers of the residues in the set L_{c}, such that these rotamer are in the set {lᵢ:i εL; rⱼ:j εR}. By definition of the tree decomposition, all the residues in L_{c} are in {L∪R}. In order to calculate the energies of the internal nodes, the nodes must be traversed in leaf to root order. Since the root has no parent, it has no set L. Its energies are given by

\tilde{E}ᵣₒₒₜ(r)=E_{R}(r)+Eₛ(r)

In the last part of the algorithm the nodes of the tree-decomposition are traversed in the root-to-leaves order to assemble the global assignment of rotamers for the cluster being processed. For any node except the root there is a local partial solution that lets for obtaining an optimal assignment of rotamers of R for any rotamer assignment of rotamers over L. But by construction L belongs to the parent bag, which means that the optimal assignment over all residues in some bag can be easily retrieved if it is know the optimal assignment at the parent node. Thus there is a recursive procedure that gradually extends an assignment for the entire cluster starting from the root node:

$${ɛ}_{root} = {min}_{r ∈ {R}_{i}} {\tilde{E}}_{root} ( r )$$

$${r}_{root} = {argmin}_{r ∈ {R}_{i}} {\tilde{E}}_{root} ( r )$$

For each child c of the root, the assignment of rotamers to the residues in Rc may be made, given the assignment of rotamers of the root, and the minimum energy added to the total:

r_{c}=rₘᵢₙ(l_{c})

εₘᵢₙ←εₘᵢₙ+\tilde{E}_{R}(l_{c},r_{c})

where the assignments in l_{c} are already known from rᵣₒₒₜ. The rotamers are assigned and the energies updated for each child of each c, and so on, following from roots to all leaves in a depth-first search.

The actual search for the local solution is done via exhaustive direct enumeration, which is quite affordable if the product of rotamer numbers within the corresponding bag is not very large. The number of possible rotamers assignments over a particular bag may be referred to as local complexity of the node. The sum of the local complexities of all nodes gives the overall computational complexity of the optimization. Typically tree-decompositions of smaller width yield lower complexity. In order to limit the time required by SCWRL4 for a single rotamer assignment an upper bound for the overall complexity of 108 can be introduced. If the actual complexity exceeds this bound then the optimization is treated as not tractable and SCWRL4 returns to the graph construction step (edge decomposition and DEE) by doubling the value of the edge decomposition threshold. This process continues until a solution is found.

### Outputting the Results.

The optimization resolves both the minimal total energy of the entire model and the corresponding assignment of rotamers. The SCWRL4 executable saves the resolved optimal conformation of the whole protein model into PDB file. The corresponding value of the total energy is printed into the standard output, which can be redirected to a file for further analysis. If the task was set up and solved via the API of the SCWRL4 library then the corresponding workspace with or without modifications can be used in forthcoming calculations.

### Training and Test Sets.

A training set of proteins was constructed for optimizing the parameters and procedures, and a separate testing set for reporting the accuracy of SCWRL4. Because it was desirable to use electron density calculations to estimate the reliability of side-chain coordinates, it was started with the list of PDB entries with electron densities available from the Uppsala Electron Density Server (Kleywegt et al. (2004) Acta Crystallogr D Biol Crystallogr., 60:2240-2249), generally those with deposited structure factors. Entries with ligands other than water were removed, so that side chains could be predicted without requiring charges, hydrogen positions, or van der Waals radii of ligands. This set was culled using the PISCES server (Wang et al. (2003) Bioinformatics, 19:1589-1591; Wang et al. (2005) Nucleic Acids Res., 33:W94-98) at maximum mutual sequence identity 30%, ≦1.8 Å resolution, and maximum R-factor of 20%. Because it was planned to optimize the energy function by predicting side chains in the crystal form, it was checked whether the CRYST1 records and scale matrices produced viable crystals. Some entries were removed that produced extensive clashes of protein atoms when crystal neighbors of the asymmetric unit were constructed (e.g. PDB entry 1RWR). The resulting list of proteins was broken up into the training and testing set, with the training set consisting of monomeric asymmetric units to speed the optimization procedures described below.

For all complete side chains in the resulting protein lists, the geometric mean of the electron density was calculated, as described previously (Shapovalov et al. (2007) Proteins, 66:279-303). In this prior work, low values of mean electron density were correlated with non-rotameric side chains and conformationally disordered side chains. For each residue type, mean electron densities for the training and testing sets were sorted and turned into percentiles, with 0% for the lowest electron density side chain and 100% for the highest.

For both sets, the program SIOCS was used available with SHELX (Sheldrick, G. M. (2008) Acta Crystallogr A, 64:112-122) to resolve the ambiguity in the flip state of Asn and His χ₂ and Gln χ₃. SIOCS uses hydrogen bonding and crystal contacts to indicate whether these side chains are correctly placed in crystal structures, or if it is likely that the terminal dihedrals should be flipped by 180°.

In crystal mode, calculations were performed on the asymmetric unit with inclusion of interactions with crystal neighbors. However, accuracy was only assessed on one protein of the asymmetric unit, as provided by PISCES.

### Optimization of the Energy Parameters.

The accuracy of SCWRL4 depends on the choice of rotamer library, the non-bonded energy functions and their parameters, the parameters used in the flexible rotamer model (FRM), and several other procedural choices. First, it was needed to decide on an objective function to be optimized. There are a number of possible choices, including RMSD, percent χ₁ correct within some threshold (typically 40° in previous literature), percent χ₁₊₂ correct, and percent of side chains correct (side chains with all χ angles within some threshold). It was decided to use the average absolute accuracy. For a side-chain type such as Lys, this is an average of percent χ₁, percent χ₁₊₂, percent χ₁₊₂₊₃, and percent χ₁₊₂₊₃₊₄ correct:

$$P {C}_{Lys} = 100 \frac{{N}_{1} + {N}_{12} + {N}_{123} + {N}_{1234}}{4 {N}_{Lys}}$$

where N₁₂ for instance is the number of lysine side chains with both χ₁ and χ₂ correct within 40°. This value gives added weight to the more reliably determined degrees of freedom closer to the backbone. To obtain an accuracy across all side-chain types, weight PC_{Xxx} for each amino acid type by its frequency:

$$PC = \frac{{\sum }_{Res} {N}_{Res} {PC}_{Res}}{{\sum }_{Res} {N}_{Res}}$$

A large number of parameters that can be optimized. First, the χ-angle deviations δᵢ for the sub-rotamers can be set individually for each dihedral degree of freedom. SCWRL4 uses a constant times the standard deviation provided in the rotamer library, where the constant is specific for each degree of freedom for each side-chain type:

δᵢ=cᵢσᵢ

The “temperature” used is also optimized in the FRM procedure separately for each amino acid type. For calculation of pairwise rotamer-rotamer energies the temperature is taken as the arithmetic average of the temperatures of the corresponding amino-acid types. The last parameter is the coefficient in front of the rotamer library term which balances the influence of the static frame and the rotamer library in the self-energy of a rotamer. Thus for every amino acid type l+2 parameters were obtained, where l is the number of χ angles required to specify the conformation of the side-chain of a certain amino-acid type. These parameters form a 78-dimensional space that can be searched to improve the quality of prediction. In addition, there are also the hydrogen bond parameters and the atomic radii that can be optimized to improve the prediction accuracy.

A special technique was used to perform the optimization in the 78-dimentional space of the parameters concerned. The main idea is similar to classical block-coordinate descent methods (Briggs et al. (2000) A Multigrid Tutorial, Philadelphia: SIAM), where on each iteration an optimum along one or several axes is resolved. In this case, the search space of any iteration consists of the parameters of a single amino-acid type, while the objective function is maximized within some vicinity of the current values of these parameters. Every iteration updates only the parameters associated with the corresponding amino-acid type while the parameters of the other amino-acid types remain unchanged. In this method, any iteration updates the parameters in a way that increases the value of the objective functions, otherwise keeping the parameters unchanged. Changing the amino-acid types from one iteration to another will impose a sequence of points in the original 78-dimensional space along which the value of the objective function will gradually increase (or at least will remain unchanged). Iterations are grouped into rounds—a randomly shuffled sequence of 18 iterations, which contains all residue types except ALA and GLY. The experiments showed that a few rounds are typically enough to find some (not necessarily global) maximum of the objective function. The approximate solution of this constrained optimization task was obtained using a special technique.

A key advantage of the protocol is that it lets the utilization of specific properties of the internal structure of the rotamer assignment procedure. When the flexible rotamer model is enabled, the most CPU-consuming part of the whole prediction is the calculation of the interaction graph and especially the calculation of the pairwise energies. However for some pair of amino acids, if neither of the rotamers has been changed then the current energy of the pairwise interaction between them is valid and does not need to be recalculated. Conversely, changing the parameters of some amino acid type affects only the rotamers of that type and their interactions with rotamers of any other type. During a single iteration only side chains of one amino-acid type are modified and so most of the interaction graph can be preserved while only a minor part has to be recomputed. Moreover if only the weight of the rotamer library's energy term is changed, then neither the static frame energies nor the energies of pairwise interactions have to be recomputed. The parameters were optimized such that all side chains were in their crystal environment, interacting with crystal neighbors of the asymmetric unit. This is particularly important for polar side chains with contacts between asymmetric units.

### Library.

SCWRL4 has been redesigned as a library, so that its optimization engine can be used in other scenarios such as protein design. It therefore utilizes a delayed computation model. At first the model data (referred to as the workspace) is defined. This means specifying the location of amino-acid residues with appropriate rotamers via calls to functions of the library. After that the calling program uses the SCWRL4 engine in the library to derive the optimal assignment of rotamers. This will request that SCWRL4 calculate all the required energies and perform combinatorial optimization after which for each residue one of its rotamer will be marked as optimal. This information can then be used in other applications. The SCWRL4 library keeps the model alive for further usage even after the optimal assignment has been found. This means that after the optimal rotamers have been resolved, some modifications can be introduced into the model and the optimization requested again. In this case SCWRL4 will recalculate only those energies that need to be modified due to changes in the model, while for energies between persistent objects it will use cached values.

### Results

### Training and Testing Sets.

Separate training and testing sets of 100 and 379 proteins respectively were used to optimize and test various parameters and algorithmic choices for development of SCWRL4. The resolution cutoff for both sets was 1.8 Å, and the maximum mutual sequence identity was 30%. All calculations below are performed either on the asymmetric unit or using crystal symmetry, although in each case the accuracy results are compiled on sets consisting of only one chain of each sequence.

### Accuracy of SCWRL4.

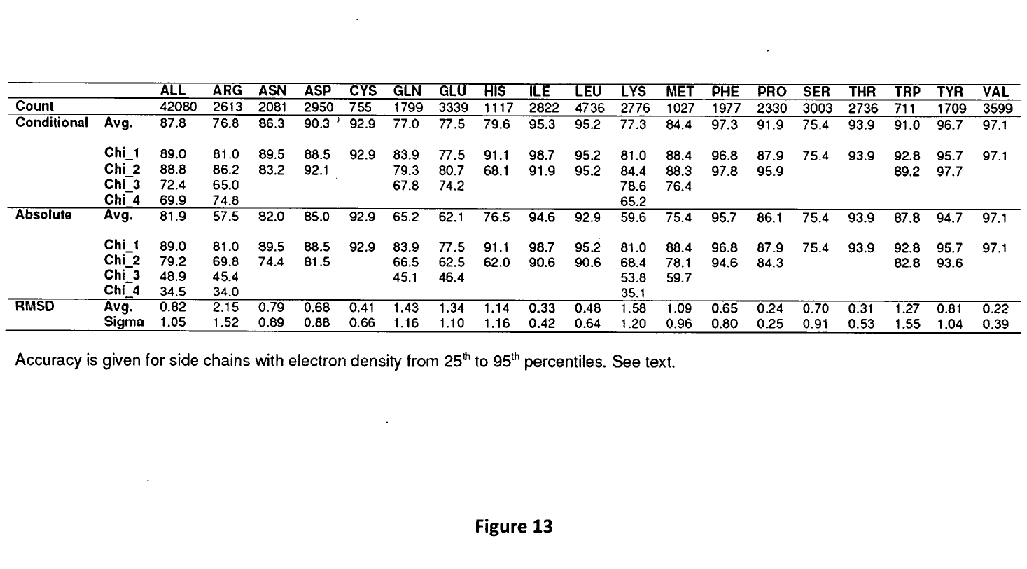

The overall accuracy of SCWRL4 is presented in Table 1 (FIG. 13) for those side chains with electron density between the 25 and 95 percentiles (see Methods). The accuracy is reported in three different ways. First, for each side-chain type, the conditional accuracy for each dihedral degree of freedom, χᵢ, is reported. This is the percent of χᵢ that is correct, given that the χ angles closer to the backbone, χᵢ₋₁, . . . , χ₁ are also correct. So for instance, for Met, for those residues with both χ₁ and χ₂ correct, 76.4% have χ₃ correct. The column “ALL” counts only those residue types with that degree of freedom. Second, for each side-chain the absolute accuracy at each degree of freedom is the percentage of all residues of that type such that χᵢ, . . . χ₁ are all correct. So for instance, for Met, 59.7% of all residues are predicted correctly for χ₁, χ₂, and χ₃. Finally, the average RMSDs are given for each side chain type (i.e., the RMSD is calculated for each residue and these values are averaged, rather than calculating an RMSD over all coordinates for a given residue type across all proteins).

The numbers most frequently cited for side-chain prediction accuracy are the χ₁ and χ₁₊₂ rates over all side-chain types, where χ₁₊₂ is the absolute accuracy at the χ₂ degree of freedom in Table 1 (FIG. 13). These values are 89.0% and 79.2% respectively for the 25-95th percentiles of electron density. For all side chains, these values fall to 86.1% and 74.8%. For 9 of 18 residue types, the χ₁ accuracies exceed 90%, and these are predominantly the aliphatic and aromatic residue types. Ser is the most difficult to predict, with an accuracy rate of 75.4%.

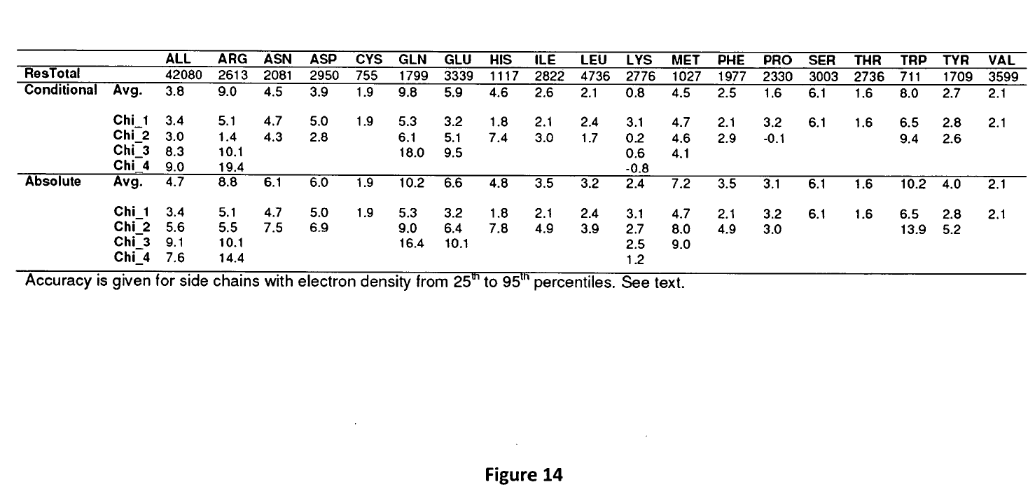

In Table 2 (FIG. 14), the improvement in prediction accuracy of SCWRL4 over SCWRL3 is shown for each residue type and for the conditional and absolute accuracy measures. The overall improvement in χ₁ accuracy is 3.4% (SCWRL4 accuracy—SCWRL3 accuracy on the same test set of 379 proteins). The largest improvements are in Trp, Gln, Glu, Met, Asp, Asn, and Ser, all of which exceed 6% improvement in total absolute accuracy.

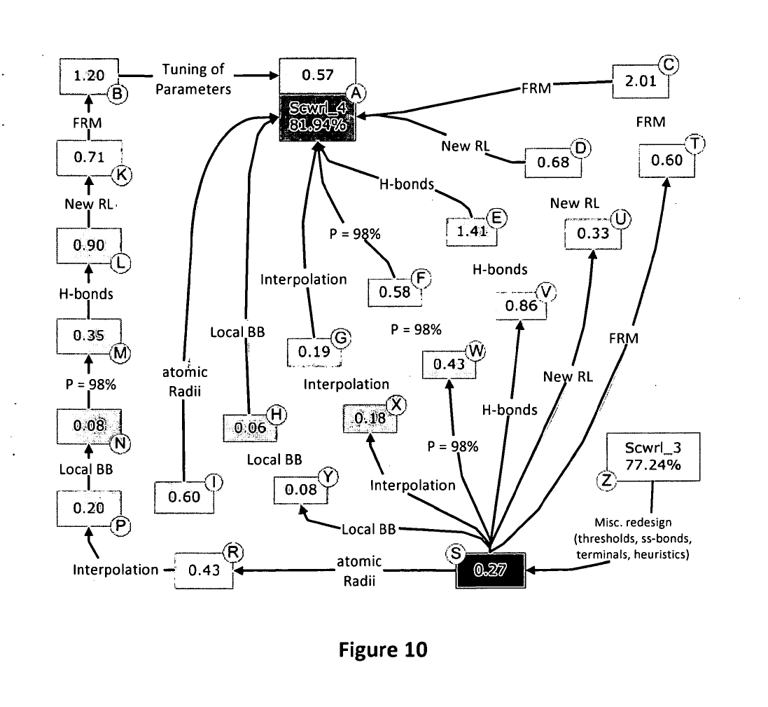

The improvement in accuracy in SCWRL4 over SCWRL3 was achieved through a number of different changes in the sampling space, the energy function, and the algorithm. Each of these feature changes was chosen and/or optimized on the basis of improvement in the training set of 100 proteins. In FIG. 10, the effects of each change added to SCWRL3 are shown (boxes R through T) or each change removed from the final SCWRL4 protocol (boxes A through I). The figure demonstrates that the effect of each feature is context-dependent; that is, the effect is different when added to SCWRL3, which contains none of the new features vs. when it is removed from the final SCWRL4, which contains all of the new features. The directed graph leading from SCWRL3 to SCWRL4 along the outside of the figure shows the improvements as each feature is added consecutively. The most important changes include the flexible rotamer model, the new rotamer library of the instant invention, the addition of a hydrogen bonding function, changes in the atomic radii used, and using a larger percentage of the rotamer library (the top 98% of rotamer density, instead of 90% as used in SCWRL3).

### Prediction of Side-Chain Conformation in Crystals.

Consideration of crystal symmetry in side-chain conformation prediction in SCWRL4 was enabled. This is accomplished by determining the interaction graph in the context of neighboring chains to the asymmetric unit within the crystal. Thus, a residue in the graph may have a neighbor on the other side of the protein, if that residue makes contact with that residue in a crystal neighbor. Crystals were built and neighbors determined as described in previous work (Xu et al. (2008) J. Mol. Biol., 381:487-507). It is of interest to determine the effect on prediction accuracy when crystal symmetry is taken into account. It should be noted that this is a bona fide prediction within the crystal, since the side chains in the neighboring asymmetric units have the same conformations as the asymmetric unit whose structure is being predicted.

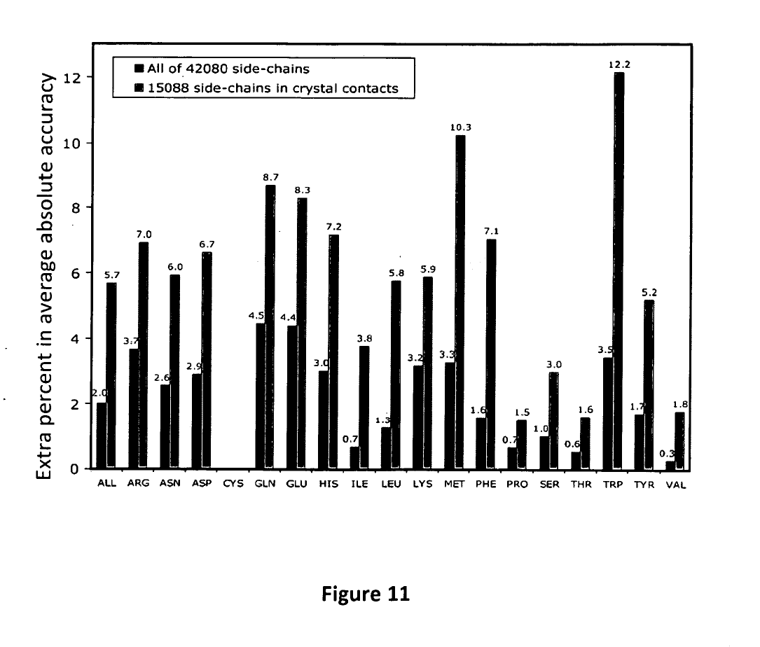

In FIG. 11, the improvement in accuracy is shown for all side chains and for those in crystal contacts when the crystal symmetry feature is enabled. The accuracy values shown are average absolute accuracy, which are averages of the χ₁, χ₂, χ₃, and χ₄ absolute accuracies shown in Table 2 (FIG. 14). Improvement occurs for all side-chain types except Cys, which is not affected by the crystal neighbors. Among all side chains, not just those in crystal contacts, the effect is strongest for those most likely to be on the surface, in particular the longer side chains, Arg, Lys, Glu, and Gln. However, when other side chain types are in crystal interfaces, their accuracy is also strongly affected by the presence of the crystal neighbors. This is especially true for Trp and Met. The χ₁ and χ₁₊₂ accuracy in the crystal for side chains with electron densities in the 25-95% percentiles are 90.5% and 82% respectively. For all side chains, the values are 87.4% and 77.1%

Prediction of Side-Chain Conformation vs. Electron Density.

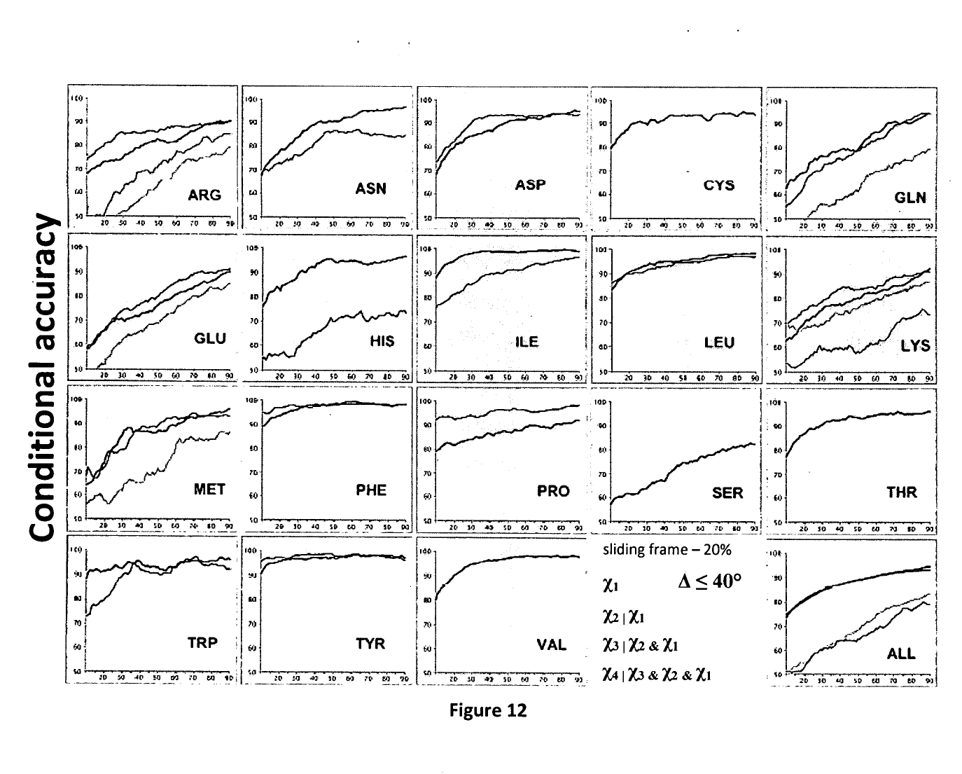

It has been previously shown that rotameric side chains have higher electron density than non-rotameric side chains, and that non-rotameric side chains are likely to be disordered in a manner similar to side chains that can be shown to exist in more than one χ₁ rotamer in the crystal (Shapovalov et al. (2007) Proteins 66:279-303). Since SCWRL4 predicts only one conformation per side chain, it seems likely that side chains with lower electron density should be harder to predict, since they may be placed in only a portion of the observable electron density. In FIG. 12, the conditional accuracy for each degree of freedom as a function of the percentile of electron density of the entire side chain is shown. Each point represents a 20-point wide bin of percentiles. Low percentiles correspond to low electron density, disordered side chains, and high percentiles correspond to high electron density, well-ordered side chains. For all side-chain types and all degrees of freedom, the conditional accuracy rises with increasing electron density. For some degrees of freedom, this is only true at low electron density, and the accuracy plateaus above the 30th percentile. But for the longer side chains, the increase in accuracy extends from percentiles of 0-20 to 80-100. For all side chains, the χ₁ accuracy and χ₂ conditional accuracy increase from 75% at the lowest electron density to over 90% at the highest electron density. The χ₃ and χ₄ conditional accuracies increase from 50% to about 80% from lowest to highest electron density.

### Discussion

While the process of sequence-structure alignment is well represented by many available web servers and downloadable programs, relatively few programs exist for producing three-dimensional coordinates from target-template alignments (Wang et al. (2005) Proteins). The MolIDE program was previously developed that takes an input target sequence and produces alignments of this target sequence to available homologous proteins of known structure (Canutescu et al. (2005) Bioinformatics, 21:2914-2916; Wang et al. (2008) Nature Protocols). In a graphical environment, it is then possible to use the SCWRL3 program to produce a model of the target sequence retaining the input sequence numbering. SCWRL4 may perform the same function within the existing MolIDE, but with higher accuracy than SCWRL3. It may also be used in existing web servers that perform searches for remote homologues such as FFAS03 (Jaroszewski et al. (2005) Nucleic Acids Res., 33:W284-288). Using the rigid rotamer model, SCWRL4 is faster than SCWRL3. With the flexible rotamer model, it is somewhat slower, by a factor of about 7, but more accurate. SCWRL4 has a similar ease of use, and therefore will function in similar environments as SCWRL3, but with higher accuracy.

There are a number of potential sources of disagreement between predicted side-chain conformations based on native backbones and experimental structures. Herein, several of these have been explored. The first and most obvious is the scoring function that must realistically represent the physical forces that position side chains in proteins. The improved rotamer library that SCWRL depends on, especially in those degrees of freedom that are not strictly rotameric. These include the amide and carboxylate moieties of Asn, Asp, Glu, and Gln, and the aromatic rings of Phe, Tyr, His, and Trp. Second, issues of sampling have been explored by including conformations near the rotameric positions using the flexible rotamer model approach of Mendes et al. (Mendes et al. (1999) Proteins, 37:530-543). Each of these issues can be explored further, for instance by including solvation energy terms as well as continuous dihedral angle minimization, as performed in Rosetta (Rohl et al. (2004) Methods Enzymol., 383:66-93). For the latter, the new rotamer libraries affords an opportunity by providing continuous energy functions as a function of the side-chain χ dihedral angles, independent of rotamer definitions.

Two other aspects of side-chain prediction have been explored that affect the overall accuracy. The first of these comprise the interactions of side chains within the crystal. For the first time, a side-chain prediction program has been developed that can account for crystal symmetry, that is, predicting the conformation of all side chains within the crystal. Increases in accuracy of almost all side-chain types are found, especially for those most likely to be in crystal contacts. Improvement in the crystal is interesting for two reasons. First, if side-chain prediction is used for molecular replacement or structure refinement, then the ability to consider the crystal symmetry will be very useful. Second, this is some indication that the prediction of side-chain conformation in protein-protein interfaces with SCWRL4 is likely to be significantly better than for side chains on the surface but not in protein interfaces.

The second important issue is the apparent disorder of many side chains in crystals that have been studied previously (Shapovalov et al. (2007) Proteins, 66:279-303). It has been shown that prediction accuracy monotonically improves with increasing electron density, such that side chains that are clearly positioned in one conformation in the crystal are the easiest to predict. The SCWRL4 algorithm is sufficiently fast that it is possible to estimate the energies of each rotamer of each side chain by predicting the conformations of the remaining side chains. In this way, concerted changes in conformations may be accounted for.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of generating a backbone-dependent rotamer library, said rotamer library comprising a set of allowable rotameric conformations for a set of amino acids or analogs thereof, said method comprising:
(a) determining rotamer probabilities as a function of φ and ψ by using von Mises kernels in adaptive kernel density estimates;
(b) determining χ angle means and variances by using non-parametric kernel regression estimates; and
(c) determining the backbone-dependent probability distributions of non-rotameric degrees of freedom using a combination of data-adaptive kernel density estimates for the side-chain degree of freedom and query-dependent kernel density estimates for the backbone degrees of freedom.

2. The method of claim 1, further comprising a Bayesian prior comprising the product of a r₁ backbone-dependent density estimate and backbone-independent conditional probabilities for the other degrees of freedom.

3. A backbone-dependent rotamer library generated by the method of claim 1.

4. A method for generating an optimized structure of at least one polypeptide, said method comprising the steps of:
(a) providing the backbone structure of said at least one polypeptide;
(b) utilizing the rotamer library of claim 3 to establish a group of potential rotamers for at least one variable residue position in said polypeptide; and
(c) analyzing the interaction of each of the rotamers obtained in step b) with at least part or all of the remainder of the polypeptide structure,
thereby generating an optimized structure of the side chains of the at least one polypeptide.

5. The method of claim 4, wherein step a) further comprises the coordinates of a ligand of cofactor and the resultant optimized structure of the polypeptide is in the presence of said ligand or cofactor.

6. The method of claim 5, wherein said ligand or cofactor is non-proteinaceous.

7. The method of claim 4, wherein at least one variable residue position has rotamers from at least two different amino acid side chains.

8. The method of claim 5, wherein said optimized structure of at least one polypeptide is an amino acid sequence of said at least one polypeptide having increased binding for said ligand or cofactor.

9. An optimized protein structure obtained by the method of claim 4.

10. The method of claim 8, further comprising:
d) comparing the binding affinity in vitro or in vivo of said optimized structure for said ligand or cofactor with the binding affinity of the unmodified polypeptide with said ligand or cofactor.

11. A method for determining whether an alteration in a first polypeptide modulates the binding of said first polypeptide to a second polypeptide, said method comprising:
(a) providing a set of structure coordinates for the amino acid residues of said first and second polypeptides;
(b) modeling the interaction between said first and second polypeptides;
(c) utilizing the rotamer library of claim 3 to establish a group of potential rotamers for at least one variable residue position in said first polypeptide, thereby producing a set of structure coordinates that define an altered first polypeptide;
(d) modeling the interactions of said altered first polypeptide with said second polypeptide; and
(e) determining whether said alteration inhibits or promotes the interaction between said altered first polypeptide and said second polypeptide compared to said first polypeptide and said second polypeptide.

12. An altered polypeptide sequence which exhibits enhanced interaction with a second polypeptide as identified by the method of claim 11.

13. The method of claim 11, wherein said first polypeptide is an antibody and said second polypeptide comprises an epitope recognized by said antibody.

14. The method of claim 11, further comprising:
f) comparing the binding affinity in vitro or in vivo of said altered first polypeptide for said second polypeptide with the binding affinity of said first polypeptide with said second polypeptide.
